(19) United States
(12) Patent Application Publication
(10) Pub. No.: US 2026/0098013 A1
(43) Pub. Date: Apr. 9, 2026

(54) Stereoisomer Conjugates of Dextrorphan

(71) Applicant: Zevra Therapeutics, Inc., Celebration, FL (US)

(72) Inventors: Sven Guenther, Coralville, IA (US); Travis Mickle, Celebration, FL (US); Sanjib Bera, Blacksburg, VA (US); Amarraj Chakraborty, Celebration, FL (US)

(21) Appl. No.: 19/097,327

(22) Filed: Apr. 1, 2025

Related U.S. Application Data
(60) Provisional application No. 63/573,445, filed on Apr. 2, 2024.

(51) Int. Cl. C07D 221/28; C07K 1/107; C07K 5/062

(52) U.S. Cl. CPC C07D 221/28; C07K 1/1077; C07K 5/06026; C07K 5/06052

(57) ABSTRACT

The present technology provides a class of compounds which are amino acid ester conjugates of dextrorphan, wherein the amino acids are diestereomerically pure, which have improved bioavailability. The present technology further provides methods of manufacturing the same. The present technology still further provides pharmaceutical compositions comprising said compounds and methods of treating diseases or conditions using the same.

### RELATED APPLICATIONS

This application claims priority to U.S. Provisional Application 63/573,445, filed on Apr. 2, 2024, the entire contents of which are incorporated by reference herein.

### BACKGROUND OF THE INVENTION

Dextrorphan ((+)-17-methylmorphinan-3-ol) is the (+)-isomer and one of two enantiomers of 17-methylmorphinan-3-ol. The other enantiomer is levorphanol ((−)-17-methylmorphinan-3-ol). A 1:1 mixture of both enantiomers (dextrorphan and levorphanol) is referred to as racemorphan.

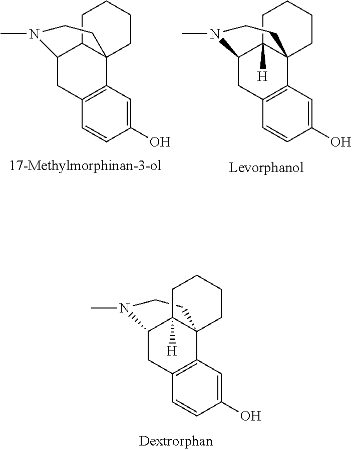 [SMILES: CN1CCC23CCCCC2C1Cc1ccc(O)cc13 ; CN1CC[C@]23CCCC[C@H]2[C@H]1Cc1ccc(O)cc13 ; CN1CC[C@@]23CCCC[C@@H]2[C@@H]1Cc1ccc(O)cc13]

Dextrorphan is a narcotic analgesic, which interacts predominantly with receptors in the central nervous system (CNS). It is also an active metabolite of dextromethorphan that forms after O-demethylation by CYP2D6. Dextrorphan has a wide range of pharmacological activities including weak μ-opioid agonism, (μ-opioid receptor (MOR)) and κ-opioid receptor agonism (KOR), as well as σ receptor agonism. Dextrorphan is also an NMDA (N-methyl-D-aspartate) receptor antagonist and a reuptake inhibitor of serotonin (SRI) and norepinephrine. In addition, dextrorphan is an antagonist of the α₃β₄, α₄β₂ and α₇ nicotinic acetylcholine receptors, and the glycine receptor. Furthermore, dextrorphan blocks the L-Type voltage-gated calcium channel (LVGCC). This multimodal pharmacological profile may be effective for the treatment of CNS conditions including, but not limited to, pain, cough, neuropathic pain, cancer pain, opioid-induced hyperalgesia, pain syndromes that are refractory to other analgesic medications, post-therapeutic neuralgia, depression, narcolepsy and hyperalgesia.

Racemates of amino acid conjugates of dextrorphan are known to exhibit lower bioavailability compared to unconjugated dextrorphan salts, as reported in U.S. Pat. No. 11,214,544, herein incorporated by reference.

However, there still exists a need for compositions of dextrorphan that provide increased dextrorphan bioavailability at lower dosages with side effects that are less frequent and/or less severe.

### BRIEF SUMMARY OF THE INVENTION

The present technology utilizes conjugation of dextrorphan with certain stereoisomers of amino acids and/or acyl amino acids to improve the bioavailability of dextrorphan over optically inactive amino acids. The present technology discloses amino acid ester conjugates of dextrorphan with superior bioavailability compared to unconjugated dextrorphan salts capable of meeting this need. The present technology further provides amino acid conjugates of dextrorphan with improved Central Nervous System (CNS) effect including but not limited to pain, cough, neuropathic pain, cancer pain, opioid-induced hyperalgesia, pain syndromes that are refractory to other analgesic medications.

In a further aspect, the present technology provides an ester conjugate of dextrorphan and at least one chiral amino acid, wherein the conjugate has at least two or more chiral centers and the amino acid is optically active. In another aspect of the present technology is provided an ester conjugate of dextrorphan and at least one chiral amino acid, wherein the resulting ester conjugate has at least two or more chiral centers.

The present technology also provides methods of delivering dextrorphan as chiral amino acid ester conjugates that release the dextrorphan following oral administration with higher bioavailability (i.e., increased AUC) and/or with an extended-release release profile that exhibits delayed peak dextrorphan plasma concentrations (Cₘₐₓ) or longer time to maximum dextrorphan plasma concentrations (Tₘₐₓ) compared to unconjugated dextrorphan when administered at equimolar doses.

The compounds and conjugates of this disclosure (aka prodrugs) may be administered alone, or combined with other CNS agents, for the treatment of CNS conditions, including, but not limited to, pain, cough, neuropathic pain, cancer pain, opioid-induced hyperalgesia, pain syndromes that are refractory to other analgesic medications, post-therapeutic neuralgia, depression, major depressive disorder, persistent depressive disorder, bipolar disorder, seasonal affective disorder, post-traumatic stress disorder (PTSD), Alzheimer's disease, anesthesia, benzodiazepine withdrawal, traumatic brain injury, stroke, Parkinson's disease, Huntington's disease, pseudobulbar affect, narcolepsy and hyperalgesia.

In another aspect, the present technology provides methods to synthesize chiral amino acid ester conjugates of dextrorphan. Alternatively, the present technology provides a method to synthesize N-acyl amino acid ester conjugates of dextrorphan.

In another aspect the present technology provides a method of treatment for CNS disorders comprising to a patient in need thereof a pharmaceutically effective amount of a chiral amino acid ester conjugate of dextrorphan, or a pharmaceutically acceptable salt thereof.

In an aspect, the present technology relates to a compound having a structure of Formula I:

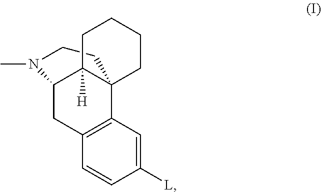

where L is A¹-G¹ or A¹-A²-G², and A¹ and A² are independently selected optically active amino acids, wherein at least one of A¹ and A² has a chiral center, G¹ is an acyl group, and G² is an acyl group or absent; or a pharmaceutically acceptable salt thereof.

In another aspect, the present technology relates to a compound having a structure of Formula I:

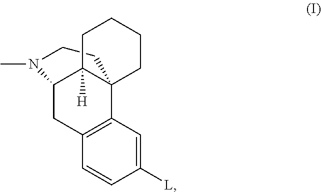

where L is A¹-G¹ or A¹-A²-G², and A¹ and A² are independently selected amino acids, wherein at least one of A¹ and A² has a chiral center and is optically active, G¹ is an acyl group, and G² is an acyl group or absent; or a pharmaceutically acceptable salt thereof.

In an aspect, the present technology relates to a compound having a structure of Formula I, wherein the at least one chiral amino acid is a standard amino acid, a non-standard amino acid, or a synthetic amino acid. Alternatively, the at least one chiral amino acid is a standard amino acid.

In an aspect, the present technology relates to a compound having a structure of Formula I, wherein the standard amino acid is selected from the group consisting of isoleucine, valine, or alanine. In a further aspect the standard amino acid is selected from D-isoleucine, D-valine, and L-alanine. In a still further aspect, A¹ is L-alanine and A² is L-alanine. Alternatively, A¹ is D-valine and A² is D-valine.

In an aspect, the present technology relates to a compound having a structure of Formula I, wherein the compound having a structure of Formula I has a structure of Formula II:

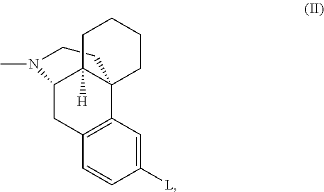

where L is A¹-G¹, A¹ is an optically active amino acids, wherein the optically active amino acid has a chiral center, and G¹ is an acyl group; or a pharmaceutically acceptable salt thereof.

In an aspect, the present technology relates to a compound having a structure of Formula I, wherein the compound having a structure of Formula I has a structure of Formula II, where the chiral amino acid is a standard amino acid, a non-standard amino acid, or a synthetic amino acid. Alternatively, the chiral amino acid is a standard amino acid.

In an aspect, the present technology relates to a compound having a structure of Formula I, wherein the compound having a structure of Formula I has a structure of Formula II, wherein the standard amino acid is selected from the group consisting of isoleucine, valine, or alanine. In a still aspect, the standard amino acid is selected from D-isoleucine, D-valine, and L-alanine.

In an aspect, the present technology relates to a compound having a structure of Formula I, wherein the compound having a structure of Formula I has a structure of Formula III:

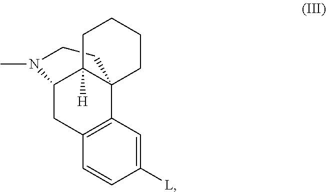

where L is A¹-A²-G², and A¹ and A² are independently selected optically active amino acids, wherein at least one of A¹ and A² has a chiral center, and G² is an acyl group or absent; or a pharmaceutically acceptable salt thereof.

In another aspect, the present technology relates to a compound having a structure of Formula I, wherein the compound having a structure of Formula I has a structure of Formula III:

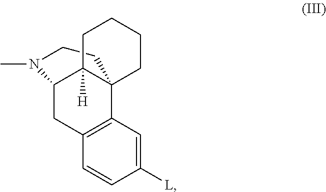

where L is A¹-G¹ or A¹-A²-G², and A¹ and A² are independently selected amino acids, wherein at least one of A¹ and A² has a chiral center and is optically active, G¹ is an acyl group, and G² is an acyl group or absent; or a pharmaceutically acceptable salt thereof.

In an aspect, the present technology relates to a compound having a structure of Formula III, where the at least one chiral amino acid is a standard amino acid, a non-standard amino acid, or a synthetic amino acid. In a further aspect, A¹ is L-alanine and A² is L-alanine. In a still further aspect, A¹ is D-valine and A² is D-valine.

In an aspect, the present technology relates to a composition comprising: a therapeutically effective amount of a compound having a structure of Formula I:

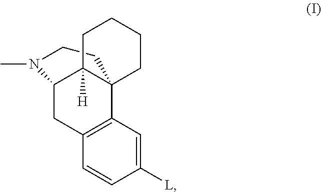

where L is A¹-G¹ or A¹-A²-G², and A¹ and A² are independently selected optically active amino acids, wherein at least one of the A¹ and/or A² has a chiral center, G¹ is an acyl group, and G² is an acyl group or absent; or a pharmaceutically acceptable salt thereof. Alternatively, A¹ and A² are independently selected amino acids, wherein at least one of A¹ and A² has a chiral center and is optically active.

In an aspect, the present technology relates to a composition comprising: a therapeutically effective amount of a compound having a structure of Formula I, where the chiral amino acid is a standard amino acid, a non-standard amino acid, or a synthetic amino acid. Alternatively, the chiral amino acid is a standard amino acid.

In an aspect, the present technology relates to a composition comprising: a therapeutically effective amount of a compound having a structure of Formula I, wherein the standard amino acid is selected from the group consisting of isoleucine, valine, or alanine. In a still aspect, the standard amino acid is selected from D-isoleucine, D-valine, and L-alanine.

In an aspect, the present technology relates to a composition comprising: a therapeutically effective amount of a compound having a structure of Formula I, wherein the therapeutically effective amount of the compound of Formula I ranges from about 0.1 mg to about 2000 mg per day.

In an aspect, the present technology relates to a composition comprising: a therapeutically effective amount of a compound having a structure of Formula I, wherein the composition further comprises at least one excipient selected from the group consisting of antiadherents, binders, coatings, disintegrants, gel forming agents, fillers, flavors and colors, glidants, lubricants, preservatives, sorbents and sweeteners.

In an aspect, the present technology relates to a composition comprising: a therapeutically effective amount of a compound having a structure of Formula I, wherein the composition is an oral dosage formulation. In a further aspect, the oral dosage formulation is a liquid dosage formulation selected from the group consisting of syrups, emulsions or suspensions. In a still further aspect, the oral dosage formulation is a solid dosage formulation selected from the group consisting of a sublingual, a gummy, a chewable tablet, a rapidly dissolving tablet, a tablet, a capsule, a caplet, a troche, a lozenge, an oral powder, a thin strip, an oral thin film (OTF), and an oral strip

In an aspect, the present technology relates to a composition comprising: a therapeutically effective amount of a compound having a structure of Formula I, wherein: the compound of Formula I has a structure of Formula II:

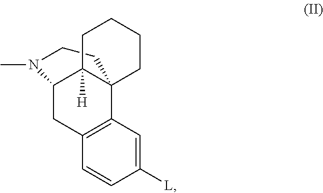

where L is A¹-G¹, and A¹ is an optically active amino acids, wherein the optically active amino acid has a chiral center, G¹ is an acyl group, or a pharmaceutically acceptable salt thereof.

In an aspect, the present technology relates to a composition comprising: a therapeutically effective amount of a compound having a structure of Formula I, wherein: the compound of Formula I has a structure of Formula II, where the chiral amino acid is a standard amino acid, a non-standard amino acid, or a synthetic amino acid. In a still further aspect, the compound of Formula I has a structure of Formula II, wherein the chiral amino acid is a standard amino acid.

In an aspect, the present technology relates to a composition comprising: a therapeutically effective amount of a compound having a structure of Formula I, wherein: the compound of Formula I has a structure of Formula II, wherein the standard amino acid is selected from the group consisting of isoleucine, valine, or alanine. In a further aspect, the standard amino acid is selected from D-isoleucine, D-valine, and L-alanine.

In an aspect, the present technology relates to a composition comprising: a therapeutically effective amount of a compound having a structure of Formula I, wherein: the compound of Formula I has a structure of Formula II, wherein the therapeutically effective amount of the compound of Formula I ranges from about 0.1 mg to about 2000 mg per day.

In an aspect, the present technology relates to a composition comprising: a therapeutically effective amount of a compound having a structure of Formula I, wherein: the compound of Formula I has a structure of Formula II, wherein the composition further comprises at least one excipient selected from the group consisting of antiadherents, binders, coatings, disintegrants, gel forming agents, fillers, flavors and colors, glidants, lubricants, preservatives, sorbents and sweeteners.

In an aspect, the present technology relates to a composition comprising: a therapeutically effective amount of a compound having a structure of Formula I, wherein: the compound of Formula I has a structure of Formula II, wherein the composition is an oral dosage formulation.

In an aspect, the present technology relates to a composition comprising: a therapeutically effective amount of a compound having a structure of Formula I, wherein: the compound of Formula I has a structure of Formula II, wherein the oral dosage formulation is a liquid dosage formulation selected from the group consisting of syrups, emulsions or suspensions.

In an aspect, the present technology relates to a composition comprising: a therapeutically effective amount of a compound having a structure of Formula I, wherein: the compound of Formula I has a structure of Formula II, wherein the oral dosage formulation is a solid dosage formulation selected from the group consisting of a sublingual, a gummy, a chewable tablet, a rapidly dissolving tablet, a tablet, a capsule, a caplet, a troche, a lozenge, an oral powder, a thin strip, an oral thin film (OTF), and an oral strip.

In an aspect, the present technology relates to a composition comprising: a therapeutically effective amount of a compound having a structure of Formula I, wherein the compound of Formula I has a structure of Formula III:

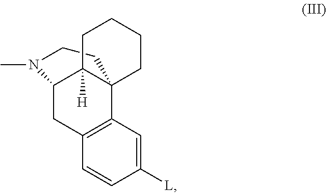

where L is A¹-A²-G², and A¹ and A² are independently selected optically active amino acids, wherein at least one of A¹ and/or A² has a chiral center, and G² is an acyl group or absent; or a pharmaceutically acceptable salt thereof. Alternatively, A¹ and A² are independently selected amino acids, wherein at least one of A¹ and A² has a chiral center and is optically active.

The composition according to claim 38, where the at least one chiral amino acid is a standard amino acid, a non-standard amino acid, or a synthetic amino acid. In a further aspect, the compound of Formula I has a structure of Formula III, wherein the at least one chiral amino acid is a standard amino acid.

In an aspect, the present technology relates to a composition comprising: a therapeutically effective amount of a compound having a structure of Formula I, wherein the compound of Formula I has a structure of Formula III, wherein A¹ is L-alanine and A² is L-alanine. In a further aspect, A¹ is D-valine and A² is D-valine.

In an aspect, the present technology relates to a composition comprising: a therapeutically effective amount of a compound having a structure of Formula I, wherein the compound of Formula I has a structure of Formula III, wherein the therapeutically effective amount of the compound of Formula I ranges from about 0.1 mg to about 2000 mg per day.

In an aspect, the present technology relates to a composition comprising: a therapeutically effective amount of a compound having a structure of Formula I, wherein the compound of Formula I has a structure of Formula III, wherein the composition further comprises at least one excipient selected from the group consisting of antiadherents, binders, coatings, disintegrants, gel forming agents, fillers, flavors and colors, glidants, lubricants, preservatives, sorbents and sweeteners.

In an aspect, the present technology relates to a composition comprising: a therapeutically effective amount of a compound having a structure of Formula I, wherein the compound of Formula I has a structure of Formula III, wherein the composition is an oral dosage formulation.

In an aspect, the present technology relates to a composition comprising: a therapeutically effective amount of a compound having a structure of Formula I, wherein the compound of Formula I has a structure of Formula III, wherein the oral dosage formulation is a liquid dosage formulation selected from the group consisting of syrups, emulsions or suspensions.

In an aspect, the present technology relates to a composition comprising: a therapeutically effective amount of a compound having a structure of Formula I, wherein the compound of Formula I has a structure of Formula III, wherein the oral dosage formulation is a solid dosage formulation selected from the group consisting of a sublingual, a gummy, a chewable tablet, a rapidly dissolving tablet, a tablet, a capsule, a caplet, a troche, a lozenge, an oral powder, a thin strip, an oral thin film (OTF), and an oral strip.

In some aspects, the present technology provides a composition comprising an ester prodrug of dextrorphan with greater bioavailability compared to unconjugated dextrorphan. In at least one aspect, the compositions/formulations of the current technology can lessen common side effects associated with unconjugated dextrorphan and similar compounds. In at least one other aspect, the compositions of the current technology can be orally administered to a human or animal patient at lower doses but with equivalent therapeutic effect compared to unconjugated dextrorphan.

In one aspect, the present technology provides a composition comprising at least one chiral amino acid ester conjugate of dextrorphan, where the at least one chiral amino acid ester conjugate comprises at least one amino acid and/or acyl amino acid, or derivatives thereof. In another embodiment the at least one amino acid and/or acyl amino acid, or derivatives thereof has a chiral center.

In an aspect, the present technology related to a method of manufacturing a compound having a structure of Formula II:

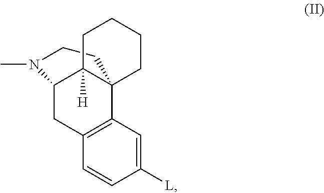

where L is A¹-G¹, A¹ is an optically active amino acids, wherein the optically active amino acid has a chiral center, and G¹ is an acyl group, and the method comprising: reacting a protected diestereomerically pure amino acid with dextrorphan in a solvent in the presence of a base for form a protected amino acid ester intermediate; and reacting the protected amino acid ester intermediate with an acid to produce a compound of Formula II.

In an aspect, the present technology related to a method of manufacturing a compound having a structure of Formula II, wherein the protected disetereomerically pure amino acid is protected by a protecting group selected from the group consisting of acetyl (Ac), tert-butyoxycarbonyl (Boc), tert-butyl (′Bu), benzyloxycarbonyl (Cbz), p-methoxybenzylcarbonyl (Moz), 9-fluorenylmethyloxycarbonyl (Fmoc), benzyl (Bn), benzoyl (Bz), phthaloyl, p-methoxybenzyl (PMB), 3,4 dimethoxybenzyl (DMPM), p-methozyphenyl (PMP), tosyl (Ts), acetamide, and pthalamide.

In an aspect, the present technology related to a method of manufacturing a compound having a structure of Formula II, wherein the protecting group is tert-butyoxycarbonyl (Boc).

In an aspect, the present technology related to a method of manufacturing a compound having a structure of Formula II, wherein the base is selected from the group consisting of 4-methylmorpholine (NMM), 4-(dimethylamino)pyridine (DMAP), N,N-diisopropylethylamine (DIPEA), lithium bis(trimethylsilyl)amide, lithium diisopropylamide (LDA), potassium tert.-butoxide, sodium tert-butoxide, lithium tert-butoxide, sodium hydride, potassium hydride, lithium hydride, an alkoxide, and a tertiary amine.

In an aspect, the present technology related to a method of manufacturing a compound having a structure of Formula II, wherein the solvent is selected from the group consisting of acetone, acetonitrile, butanol, chloroform, dichloromethane (DCM), dimethylformamide (DMF), dimethylsulfoxide (DMSO), dioxane, ethanol, ethyl acetate, diethyl ether, heptane, hexane, methanol, methyl tert.-butyl ether (MTBE), isopropanol, isopropyl acetate, diisopropyl ether, tetrahydrofuran, toluene, xylene, and water.

In an aspect, the present technology relates to a method of manufacturing a chiral amino acid ester prodrug of dextrorphan comprising a compound having Formula III:

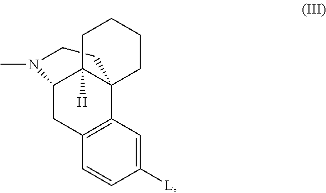

where L is A¹-A²-G², A¹ and A² are independently selected optically active amino acids, wherein at least one of A¹ and A² has a chiral center, and G² is an acyl group or absent; the method comprising: reacting a first protected diestereomerically pure amino acid with dextrorphan in a solvent in the presence of a base to produce a first intermediate; reacting the first intermediate with an acid to form a deprotected amino acid ester intermediate; reacting the deprotected amino acid ester intermediate with a second protected diestereomerically pure amino acid to produce a protected dipeptide intermediate in a second solvent in the presence of a second base; and reacting the protected dipeptide intermediate with an acid to produce a compound of Formula III.

In an aspect, the present technology relates to a method of manufacturing a chiral amino acid ester prodrug of dextrorphan comprising a compound having Formula III, wherein the protected disetereomerically pure amino acid is protected by a first protecting group selected from the group consisting of acetyl (Ac), tert-butyoxycarbonyl (Boc), tert-butyl (′Bu), benzyloxycarbonyl (Cbz), p-methoxybenzylcarbonyl (Moz), 9-fluorenylmethyloxycarbonyl (Fmoc), benzyl (Bn), benzoyl (Bz), phthaloyl, p-methoxybenzyl (PMB), 3,4 dimethoxybenzyl (DMPM), p-methozyphenyl (PMP), tosyl (Ts), acetamide, and pthalamide. Alternatively, A¹ and A² are independently selected amino acids, wherein at least one of A¹ and A² has a chiral center and is optically active.

In an aspect, the present technology relates to a method of manufacturing a chiral amino acid ester prodrug of dextrorphan comprising a compound having Formula III, wherein the protecting group is tert-butyoxycarbonyl (Boc).

In an aspect, the present technology relates to a method of manufacturing a chiral amino acid ester prodrug of dextrorphan comprising a compound having Formula III, wherein the base is selected from the group consisting of 4-methylmorpholine (NMM), 4-(dimethylamino)pyridine (DMAP), N,N-diisopropylethylamine (DIPEA), lithium bis(trimethylsilyl)amide, lithium diisopropylamide (LDA), potassium tert.-butoxide, sodium tert-butoxide, lithium tert-butoxide, sodium hydride, potassium hydride, lithium hydride, an alkoxide, and a tertiary amine.

In an aspect, the present technology relates to a method of manufacturing a chiral amino acid ester prodrug of dextrorphan comprising a compound having Formula III, wherein the solvent is selected from the group consisting of acetone, acetonitrile, butanol, chloroform, dichloromethane (DCM), dimethylformamide (DMF), dimethylsulfoxide (DMSO), dioxane, ethanol, ethyl acetate, diethyl ether, heptane, hexane, methanol, methyl tert.-butyl ether (MTBE), isopropanol, isopropyl acetate, diisopropyl ether, tetrahydrofuran, toluene, xylene, and water.

Advantages of certain embodiments of the dextrorphan prodrugs of the present technology include, but are not limited to increased oral bioavailability of dextrorphan, extended release of dextrorphan, reduced patient to patient variability in plasma concentrations compared to unconjugated dextrorphan, improved dosage forms through modifications of the physical and chemical properties of the prodrugs.

### BRIEF DESCRIPTION OF THE DRAWINGS

FIG. 1. Oral PK profiles comparing dextrorphan, 3-(N—Ac-L-Phe)-dextrorphan, and 3-(N—Ac-D-Phe)-dextrorphan.

FIG. 2. Oral PK profiles comparing dextrorphan, 3-(N—Ac-L-Ile)-dextrorphan, and 3-(N—Ac-D-Ile)-dextrorphan.

FIG. 3. Oral PK profiles comparing dextrorphan, 3-(N—Ac-L-Lys(Ac))-dextrorphan, and 3-(N—Ac-D-Lys(Ac))-dextrorphan.

FIG. 4. Oral PK profiles comparing dextrorphan, 3-(N—Ac-L-Val)-dextrorphan, and 3-(N—Ac-D-Val)-dextrorphan.

FIG. 5. Oral PK profiles comparing dextrorphan, 3-(N—Ac-L-Ala)-dextrorphan, and 3-(N—Ac-D-Ala)-dextrorphan.

FIG. 6. Oral PK profiles comparing dextrorphan, 3-(N—Ac-L-Glu)-dextrorphan, and 3-(N—Ac-D-Glu)-dextrorphan.

FIG. 7. Oral PK profiles comparing dextrorphan and 3-(L-Val-L-Val)-dextrorphan.

FIG. 8. Oral PK profiles comparing dextrorphan and 3-(L-Ala-L-Ala)-dextrorphan.

FIG. 9 Oral PK profiles comparing dextrorphan and 3-(D-Ala-D-Ala)-dextrorphan.

FIG. 10 Oral PK profiles comparing dextrorphan, 3-(D-Val-D-Val)-dextrorphan, and 3-(D-Ala-D-Val)-dextrorphan.

### DETAILED DESCRIPTION

The present technology provides compounds and compositions comprising one or more amino acids and/or acyl amino acids that are chemically conjugated to dextrorphan ((+)-17-methylmorphinan-3-ol) to form novel ester prodrugs and compositions of dextrorphan represented by Formula I:

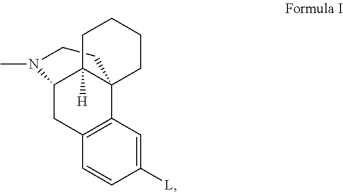

where L is A¹-G¹ or A¹-A²-G², and A¹ and A² are independently selected amino acids, wherein at least one of the A¹ and/or A² has a chiral center, G¹ is an acyl group, and G² is an acyl group or absent; or a pharmaceutically acceptable salt thereof.

Representative examples include, but are not limited to 3-(N—Ac-D-Val)-dextrorphan; 3-(N—Ac-D-Ile)-dextrorphan; 3-(N—Ac-L-Val)-dextrorphan; 3-(N—Ac-L-Ile)-dextrorphan; 3-(L-Ala-L-Ala)-dextrorphan; 3-(D-Val-D-Val)-dextrorphan; 3-(L-Val-L-Val)-dextrorphan; 3-(N—Ac-L-Lys(Ac))-dextrorphan;

### Introduction

Conventional amino acid ester conjugates of dextrorphan can have lower bioavailability. There exists a need in the art for conjugates of dextrorphan that have improved bioavailability which would allow administration at lower dosages and reduce the severity and/or frequency of side effects.

Recreational drug abuse of is a common problem and usually begins with oral doses taken with the purpose of achieving euphoria (“rush”, “high”). Over time the drug abuser often increases the oral dosages to attain more powerful “highs” or to compensate for heightened opioid tolerance. Rapid metabolism and fast duration of action of dextrorphan, contributes to its likelihood of being abused. This behavior can escalate and result in exploring of other routes of administration such as intranasal (“snorting”) and intravenous (“shooting”). In some embodiments, dextrorphan that is conjugated with a suitable ligand exhibits no rapid spikes in blood levels after oral administration that is sought by a potential drug abuser. These prodrugs may have a delayed Tₘₐₓ and possibly lower Cₘₐₓ than the parent drug and therefore lack the feeling of a “rush” when taken orally even at higher doses while still maintaining pain relief. In another embodiment, dextrorphan conjugated with appropriate ligands of this invention is not hydrolyzed efficiently when administered via non-oral routes. As a result, they do not generate high plasma or blood concentrations of released dextrorphan when injected or snorted compared to free dextrorphan administered through these routes. Furthermore, since the ligands of this invention are bound covalently to dextrorphan, the opioid is not liberated by any type of physical manipulation as it is possible, for example, by grinding up or crushing of certain kinds of formulated dextrorphan.

The inventors of the instant technology discovered that during the synthesis of amino acid conjugates of dextrorphan, the conjugation reaction produces a racemate when a stereoisomerically pure amino acid is used as the staring material. The instant technology provides a synthetic scheme that prevents the formation of racemate amino acid conjugates of dextrorphan.

The present technology unexpectedly found that the oral bioavailability of amino acid ester prodrugs of dextrorphan improved when the dose content of one diastereomer of the amino acid prodrug increased. In certain aspects of the present technology, increasing the amount of the dextrorphan prodrug containing at least one D-amino acid improved bioavailability. In other aspects of the present technology, increasing the amount of the dextrorphan prodrug containing at least one L-amino acid improved bioavailability.

Unexpectedly, the dextrorphan AUC₀₋₆ₕ was more than 5 and 8 times higher after dosing of 3-(N—Ac-D-Val)-dextrorphan·HCl and 3-(N—Ac-D-Ile)-dextrorphan·HCl, respectively, when compared to dextrorphan tartrate. However, the AUC₀₋₆ₕ of 3-(N—Ac-L-Val)-dextrorphan·HCl was significantly lower compared to dextrorphan tartrate, while the AUC₀₆ₕ of 3-(N—Ac-L-Ile)-dextrorphan HCl was only slightly lower compared to dextrorphan tartrate. The dextrorphan Cₘₐₓ was about 1.8 times higher after administration of 3-(N—Ac-D-Val)-dextrorphan·HCl, but over 6 times higher after administration of 3-(N—Ac-D-Ile)-dextrorphan·HCl when compared to dextrorphan tartrate. Conversely, the Cₘₐₓ for 3-(N—Ac-L-Val)-dextrorphan·HCl was less than 20% of the Cₘₐₓ for dextrorphan tartrate, while the Cₘₐₓ for 3-(N—Ac-L-Ile)-dextrorphan·HCl was slightly higher compared to dextrorphan tartrate.

### Definitions

“A” and “an” as it relates to the present technology, means the singular form, but includes the plural form unless clear from the context.

“About” as it related to the present technology means, as it applied to measured quantities, +/−10% of the stated measured value; for example, “about 100 mg” means 100 mg+/−10%, i.e. 90-110 mg. Unless specifically stated or obvious from context, as used herein, the term “about” is understood as within a range of normal tolerance in the art, for example, within two standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

As used herein, the term “or” means, and is used interchangeably with, the term “and/or,” unless context clearly indicates otherwise.

As used herein, the term “such as” means, and is used interchangeably with, the phrase “such as, for example” or “such as but not limited.”

As used herein, the term “subject” means a human or animal, including but not limited to a human or animal patient.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

AUC₀₋₂₄ₕ, as it relates to the present technology, is a term used in pharmacokinetics to describe the area under the curve in a plot of drug concentration in blood, serum, or plasma vs time from time=0 (or predose) to 24 hours after dosing.

AUC_{inf}, as it relates to the present technology, is a term used in pharmacokinetics to describe the area under the curve in a plot of drug concentration in blood, serum, or plasma vs time from time=0 (or predose) to infinity.

“Bioavailability,” as it relates to the present technology, means the proportion of a drug or other substance.

“Cₘₐₓ,” used hereinafter, is a term used in pharmacokinetics and refers to the maximum (or peak) plasma concentration that a drug achieves in a specified compartment or test area of the body after the drug has been administered and before the administration of a second dose.

Daily dosage, as it relates to the present technology, means the total amount of drug administered in a human or animal subject in a single day.

As used herein, the phrases such as “decreased,” “reduced,” “diminished” or “lowered” are meant to include at least about a 10% change as it relates to, for example, pharmacological activity, area under the curve (AUC), and peak plasma concentration (Cₘₐₓ). For instance, the change may also be greater than about 10%, about 15%, about 20%, about 25%, about 35%, about 45%, about 55%, about 65%, about 75%, about 85%, about 95%, about 96%, about 97%, about 98%, about 99%, or increments therein.

The use of the term “dose” means the total amount of a drug or active component taken each time by an individual subject. Once that enters the systemic circulation over time when introduced into the body.

“Patient,” as it related to the present technology, means a human or animal subject in need of treatment.

“Pharmaceutical composition” or “pharmaceutical compositions,” as it relates to the present technology, means formulations for administration to the patient including solid or liquid oral formulations, parenteral formulations, suppositories, dermal patches, and topical ointments.

“Pharmaceutically effective amount” as used herein means an amount that has a pharmacological effect.

A “pharmaceutically acceptable salt” as used herein is a salt of the conjugate of dextrorphan which, when used in a pharmaceutically effective amount, has at least one pharmacological effect.

As used herein, the term “subject” means a human or animal, including but not limited to a human or animal patient.

As known to those skilled in the art, the term “Steady State” means the state in which the overall intake of a drug is in approximate dynamic equilibrium with its elimination. At steady state, total drug exposure does not change significantly between successive dosing periods. Steady state is typically achieved following a time period about 4-5 times the half-life of a drug after regular dosing was started.

“Therapeutically effective amount” as used herein means an amount effective for treating a disease or condition.

A “therapeutically acceptable salt” as used herein is a pharmaceutically acceptable salt of the d-methylphenidate conjugate or unconjugated methylphenidate or both in the composition of the present technology, which, when used in a therapeutically effective amount, is effective for treating a disease, condition, or syndrome.

Tₘₐₓ, used hereinafter, is the term used in pharmacokinetics to describe the time at which the Cₘₐₓ is observed.

“Treating” as it related to the present technology means any of the following: (1) delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

Unless otherwise defined, all technical and scientific terms used herein have the same meaning as commonly understood by one of ordinary skill in the art to which the methods described herein belong. Any reference to standard methods (e.g., ASTM, TAPPI, AATCC, etc.) refers to the most recent available version of the method at the time of filing of this disclosure unless otherwise indicated.

### Chiral Amino Acid Conjugates of Dextrorphan

In an embodiment, the present disclosure describes a chiral amino acid ester prodrug of dextrorphan having the following general Formula I:

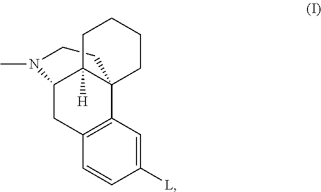

where L is A¹-G¹ or A¹-A²-G², and A¹ and A² are independently selected amino acids, wherein at least one of the A¹ and/or A² has a chiral center, G¹ is an acyl group, and G² is an acyl group or absent; or a combination thereof, or pharmaceutically acceptable salts thereof. In a further aspect, the compound has a structure of Formula II, wherein L is A¹-G¹ and G¹ is an acyl group. Alternatively, the compound has a structure of Formula III where L is A1-A1-G2 where G² is an acyl group or absent. The compounds of Formulae I, II, and III are chiral amino ester conjugates of dextrorphan.

The use of the term “dextrorphan” herein means the (+)-isomer of 17-methylmorphinan-3-ol, including all salt forms thereof. Depending on the chemical structures and specific isomers of the amino acids or acyl amino acids that are attached to dextrorphan, the resulting prodrug can be mixtures of one or more optically active isomers, wherein at least one of said isomers is optically active. In an alternative embodiment all of the isomers are optically active single isomers.

In an embodiment, the present disclosure describes a compound having the following general Formula I:

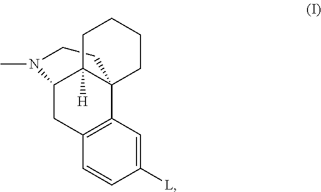

where L is A¹-G¹ or A¹-A²-G², and A¹ and A² are independently selected optically active amino acids, wherein at least one of the A¹ and/or A² has a chiral center, G¹ is an acyl group, and G² is an acyl group or absent; or a combination thereof, or pharmaceutically acceptable salts thereof.

As used herein, the term “prodrug” refers to a substance converted from an inactive form of a drug to an active drug in the body by a chemical or biological reaction. In the present technology, the prodrug is a conjugate of at least one drug, dextrorphan, and at least one amino acid or acyl amino acid, for example. Thus, the conjugates of the present technology are prodrugs and the prodrugs of the present technology are conjugates.

Prodrugs are often useful because, in some embodiments, they may be easier to administer or process than the parent drug. They may, for instance, be more bioavailable by oral administration whereas the parent drug is not. The prodrug may also have improved solubility in pharmaceutical compositions over the parent drug. An embodiment of a prodrug would be a dextrorphan conjugate that is metabolized to release the active moiety. In certain embodiments, upon in vivo administration, a prodrug is chemically converted to the biologically, pharmaceutically or therapeutically more active form of the compound. In certain embodiments, a prodrug is enzymatically metabolized by one or more steps or processes to the biologically, pharmaceutically or therapeutically active form of the compound. To produce a prodrug, a pharmaceutically active compound is modified such that the active compound will be regenerated upon in vivo administration. The prodrug is designed to alter the metabolism, pharmacokinetics, or the transport characteristics of a drug in certain embodiments, to mask side-effects or toxicity, to improve bioavailability and/or water solubility, to improve the flavor of a drug or to alter other characteristics or properties of a drug in other discrete embodiments.

“Acyl,” as it relates to the present technology means a chemical functional group with the general formula R(C═O)—, where R represents an organic group. In certain embodiments, the acyl group is a carboxylic acid. In certain aspects, the acyl group is a ketone. In certain aspects, the acyl group is an aldehyde. In certain embodiments, the acyl group is an ester. In certain embodiments, the acyl group is R(C═O)CH₃.

### Amino Acids

Amino acids of the present technology are a class of compounds which contain both a carboxylic acid and an amino functional group. In acyl amino acids, at least one acyl group is bound to at least one amino group of the amino acid. Amino acids are widespread in nature (naturally occurring), but amino acids can also be non-natural (synthetic). Amino acids can be categorized into numerous classes based on their molecular structure or formula, and many of the different classes may overlap.

Without wishing to limit the scope to one classification, the amino acids of the present technology can be grouped into the following categories: natural amino acids, synthetic amino acids, standard amino acids, non-standard amino acids, essential amino acids, conditionally essential amino acids, and non-essential amino acids. In certain aspects of the present technology, the amino acid is an essential amino acid. In other aspects of the present technology, the acid amino acid is an N-acetyl amino acid.

Amino acids are one of the most important building blocks of life. They constitute the structural subunit of proteins, peptides, and many secondary metabolites. In addition to the 22 standard (proteinogenic) amino acids that make up the backbone of proteins, there are hundreds of other natural (non-standard) amino acids that have been discovered either in free form or as components in natural products. The amino acids used in some embodiments of the prodrugs of this invention include natural amino acids, synthetic (non-natural, unnatural) amino acids, and their derivatives.

### Standard Amino Acids

There are currently 22 known standard or proteinogenic amino acids that make up the monomeric units of proteins and are encoded in the genetic code. The standard amino acids include alanine, arginine, asparagine, aspartic acid, cysteine, glutamic acid, glutamine, glycine, histidine, isoleucine, leucine, lysine, methionine, phenylalanine, proline, pyrrolysine, selenocysteine, serine, threonine, tryptophan, tyrosine and valine.

### Non-Standard Amino Acids

Non-standard amino acids can be found in proteins created by chemical modifications of standard amino acids already incorporated in the proteins. This group also includes amino acids that are not found in proteins but are still present in living organisms either in their free form or bound to other molecular entities. Non-standard amino acids occur mostly as intermediates in metabolic pathways of standard amino acids and are not encoded by the genetic code. Examples of non-standard amino acids include but are not limited to ornithine, homoarginine, citrulline, homocitrulline, homoserine, theanine, γ-aminobutyric acid, 6-aminohexanoic acid, sarcosine, cartinine, 2-aminoadipic acid, pantothenic acid, taurine, hypotaurine, lanthionine, thiocysteine, cystathionine, homocysteine, β-amino acids such as β-alanine, β-aminoisobutyric acid, β-leucine, β-lysine, β-arginine, β-tyrosine, β-phenylalanine, isoserine, β-glutamic acid, β-tyrosine, β-dopa (3,4-dihydroxy-L-phenylalanine), α,α-disubstituted amino acids such as 2-aminoisobutyric acid, isovaline, di-n-ethylglycine, N-methyl acids such as N-methyl-alanine, L-abrine, hydroxy-amino acids such as 4-hydroxyproline, 5-hydroxylysine, 3-hydroxyleucine, 4-hydroxyisoleucine, 5-hydroxy-L-tryptophan, cyclic amino acids such as 1-aminocyclopropyl-1-carboxylic acid, azetidine-2-carboxylic acid and pipecolic acid.

### Synthetic Amino Acids

Synthetic amino acids do not occur in nature and are prepared synthetically. Examples include but are not limited to allylglycine, cyclohexylglycine, N-(4-hydroxyphenyl)glycine, N-(chloroacetyl)glycline ester, 2-(trifluoromethyl)-phenylalanine, 4-(hydroxymethyl)-phenylalanine, 4-amino-phenylalanine, 2-chlorophenylglycine, 3-guanidino propionic acid, 3,4-dehydro-proline, 2,3-diaminobenzoic acid, 2-amino-3-chlorobenzoic acid, 2-amino-5-fluorobenzoic acid, allo-isoleucine, tert-leucine, 3-phenylserine, isoserine, 3-aminopentanoic acid, 2-amino-octanedioic acid, 4-chloro-β-phenylalanine, β-homoproline, B-homoalanine, 3-amino-3-(3-methoxyphenyl) propionic acid, N-isobutyryl-cysteine, 3-amino-tyrosine, 5-methyl-tryptophan, 2,3-diaminopropionic acid, 5-aminovaleric acid, and 4-(dimethylamino) cinnamic acid. The above defined prodrugs of dextrorphan can be given orally and, upon administration, release the active dextrorphan after being hydrolyzed in the body. The claimed prodrugs themselves are either not or have limited pharmacological activity and consequently may follow a metabolic pathway that differs from the parent drug. By choosing suitable amino acids and/or acyl amino acids, the release of dextrorphan into the systemic circulation can be controlled even when the prodrug is administered via routes other than oral. In one embodiment, the modified dextrorphan would release dextrorphan similar to free or unmodified dextrorphan. In a further embodiment, the modified dextrorphan would increase the oral bioavailability of dextrorphan compared to unmodified dextrorphan. In another embodiment, the modified dextrorphan would be released in a controlled or sustained manner.

The increased oral bioavailability could allow for lower dosages compared to unmodified dextrorphan while providing similar therapeutic effects.

The lower dosages and/or the controlled release of dextrorphan delivered by the prodrug can potentially alleviate certain side-effects and improve upon the safety profile of the parent drug. These side-effects may include but are not limited to dizziness, lightheadedness, drowsiness, nausea, vomiting, constipation, stomach pain, rash, difficulty urinating, difficulty breathing and fainting.

In an embodiment, the present disclosure provides a compound having a structure of Formula I, wherein the at least one chiral amino acid is a standard amino acid, a non-standard amino acid, or a synthetic amino acid. Alternatively, the at least one chiral amino acid is a standard amino acid.

In an embodiment, the present disclosure provides a compound having a structure of Formula I, wherein the standard amino acid is selected from the group consisting of isoleucine, valine, or alanine. In a further aspect the standard amino acid is selected from D-isoleucine, D-valine, and L-alanine. In a still further aspect, A¹ is L-alanine and A² is L-alanine. Alternatively, A¹ is D-valine and A² is D-valine.

In an embodiment, the present disclosure provides a compound having a structure of Formula I, wherein the compound having a structure of Formula I has a structure of Formula II:

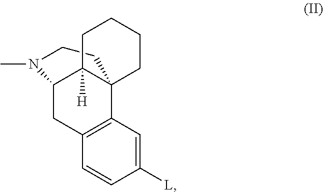

where L is A¹-G¹, A¹ is an optically active amino acids, wherein the optically active amino acid has a chiral center, and G¹ is an acyl group; or a pharmaceutically acceptable salt thereof.

In an embodiment, the present disclosure provides a compound having a structure of Formula I, wherein the compound having a structure of Formula I has a structure of Formula II, where the chiral amino acid is a standard amino acid, a non-standard amino acid, or a synthetic amino acid. Alternatively, the chiral amino acid is a standard amino acid.

In an embodiment, the present disclosure provides a compound having a structure of Formula I, wherein the compound having a structure of Formula I has a structure of Formula II, wherein the standard amino acid is selected from the group consisting of isoleucine, valine, or alanine. In a still aspect, the standard amino acid is selected from D-isoleucine, D-valine, and L-alanine.

In an embodiment, the present disclosure provides a compound having a structure of Formula I, wherein the compound having a structure of Formula I has a structure of Formula III:

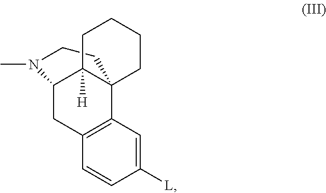

where L is A¹-A²-G², and A¹ and A² are independently selected optically active amino acids, wherein at least one of A¹ and A² has a chiral center, and G² is an acyl group or absent; or a pharmaceutically acceptable salt thereof.

In an embodiment, the present disclosure provides a compound having a structure of Formula I, wherein the compound having a structure of Formula I has a structure of Formula III:

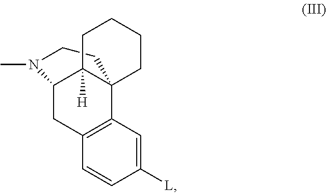

where L is A¹-G¹ or A¹-A²-G², and A¹ and A² are independently selected amino acids, wherein at least one of A¹ and A² has a chiral center and is optically active, G¹ is an acyl group, and G² is an acyl group or absent; or a pharmaceutically acceptable salt thereof.

In an embodiment, the present disclosure provides a compound having a structure of Formula I, wherein the compound having a structure of Formula I has a structure of Formula III, where the at least one chiral amino acid is a standard amino acid, a non-standard amino acid, or a synthetic amino acid. In a further aspect, A¹ is L-alanine and A² is L-alanine. In a still further aspect, A¹ is D-valine and A² is D-valine.

In an embodiment, the present disclosure provides for a compound having a structure selected from the group consisting of

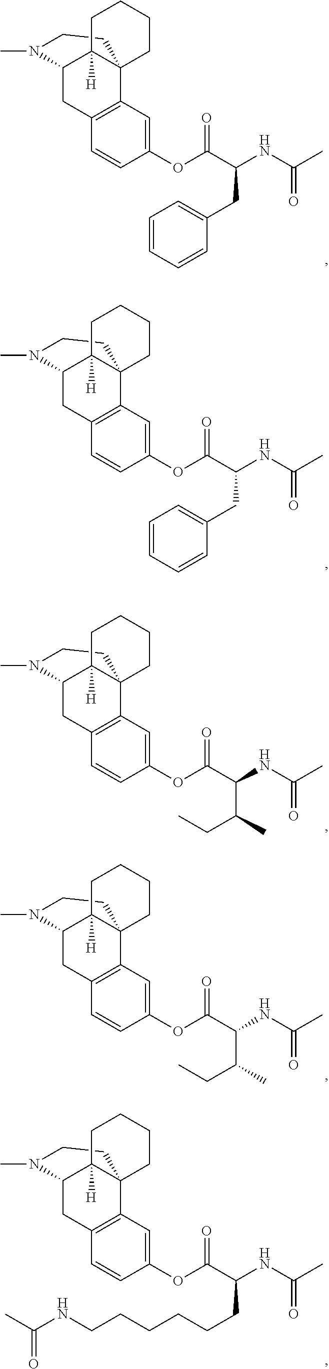

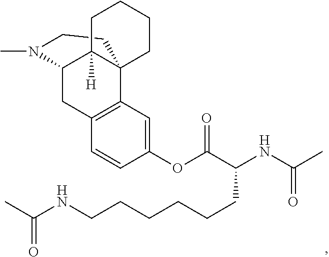 [SMILES: CC(=O)NCCCCCC[C@@H](NC(C)=O)C(=O)Oc1ccc2c(c1)[C@]13CCCC[C@@H]1[C@H](C2)N(C)CC3]

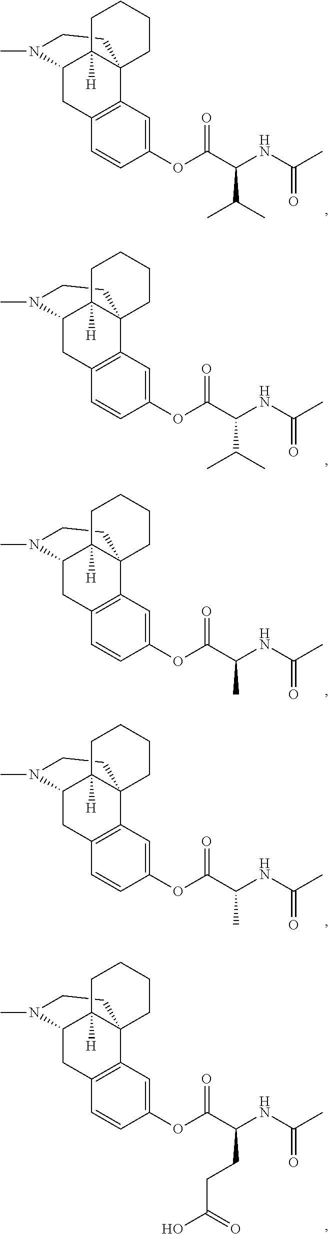

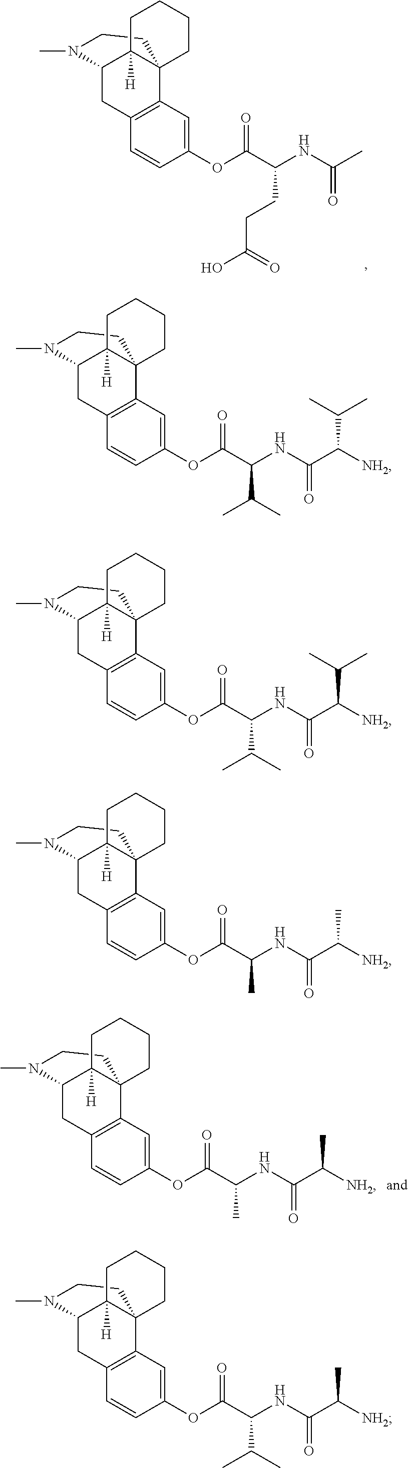

or a pharmaceutically acceptable salt thereof.

### Synthetic Schemes

Chiral amino acid conjugated dextrorphan compounds of the present technology can be prepared through an esterification reaction between enantiomerically pure amino acids and dextrorphan. In certain aspects of the present technology, the synthesis can be modified with additional chemicals that preserve the enantiomeric purity of the chiral amino acid during the esterification reaction.

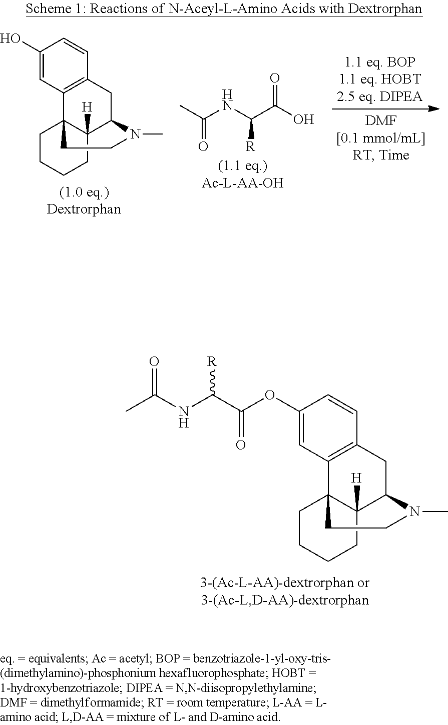

In some embodiments, one or more protecting groups may be attached to any additional reactive functional groups that may interfere with the coupling to dextrorphan. Any suitable protecting group may be used depending on the type of functional group and reaction conditions. Some protecting group suitable for use in the present technology include, but are not limited to, acetyl (Ac), tert-butyoxycarbonyl (Boc), tert-butyl (′Bu), benzyloxycarbonyl (Cbz), p-methoxybenzylcarbonyl (Moz), 9-fluorenylmethyloxycarbonyl (Fmoc), benzyl (Bn), benzoyl (Bz), phthaloyl, p-methoxybenzyl (PMB), 3,4 dimethoxybenzyl (DMPM), p-methozyphenyl (PMP), tosyl (Ts), or amides (like acetamides, pthalamides, and the like).

In other embodiments, a base may be required at any step in the synthetic scheme of prodrugs of dextrorphan of this invention. Suitable bases include, but are not limited to, 4-methylmorpholine (NMM), 4-(dimethylamino)pyridine (DMAP), N,N-diisopropylethylamine (DIPEA), lithium bis(trimethylsilyl)amide, lithium diisopropylamide (LDA), any alkali metal tert.-butoxide (e.g., potassium tert.-butoxide), any alkali metal hydride (e.g., sodium hydride), any alkali metal alkoxide (e.g., sodium methoxide), triethylamine or any other tertiary amine.

Suitable solvents that can be used for any reaction at any step in the synthetic scheme of a prodrug of dextrorphan of this invention include, but are not limited to, acetone, acetonitrile, butanol, chloroform, dichloromethane (DCM), dimethylformamide (DMF), dimethylsulfoxide (DMSO), dioxane, ethanol, ethyl acetate, diethyl ether, heptane, hexane, methanol, methyl tert.-butyl ether (MTBE), isopropanol, isopropyl acetate, diisopropyl ether, tetrahydrofuran, toluene, xylene or water.

In some embodiments, an acid may be used to remove certain protecting groups. Suitable acids include, but are not limited to, hydrochloric acid, hydrobromic acid, hydrofluoric acid, hydriodic acid, sulfuric acid, phosphoric acid, trifluoroacetic acid, acetic acid, citric acid, methanesulfonic acid, p-toluenesulfonic acid and nitric acid. For certain other protecting groups, a catalytic hydrogenation may be used, e.g., palladium on charcoal in the presence of hydrogen gas.

In some embodiments, a coupling agent may be used to facilitate the conjugation of the amino acid and/or acyl amino acid with dextrorphan or the coupling with additional amino acids and/or acyl amino acids. Suitable coupling or activating agents include, but are not limited to benzotriazole-1-yl-oxy-tris-(dimethylamino)-phosphonium hexafluorophosphate (BOP); 1-hydroxybenzotriazole (HOBT); 2-(1H-benzotriazole-1-yl)-1,1,3,3-tetramethylaminium tetrafluoroborate (TBTU); 1-[(1-(cyano-2-ethoxy-2-oxoethylideneaminooxy)dimethylaminomorpholino)]uronium hexafluorophosphate (COMU); 1-[bis(dimethylamino)methylene]-1H-1,2,3-triazolo[4,5-b]pyridinium 3-oxid hexafluorophosphate (HATU); 2-(1H-benzotriazole-1-yl)-1,1,3,3-tetramethyluronium hexafluorophosphate (HBTU); O-(1H-6-chlorobenzotriazole-1-yl)-1,1,3,3-tetramethyluronium hexafluorophosphate (HCTU); (7-azabenzotriazol-1-yloxy)trispyrrolidinophosphonium hexafluorophosphate (PyAOP); benzotriazole-1-yl-oxy-tris-pyrrolidino-phosphonium hexafluorophosphate (PyBOP); 6-chloro-benzotriazole-1-yloxy-tris-pyrrolidinophosphonium hexafluorophosphate (PyClock); 1-cyano-2-ethoxy-2-oxoethylideneaminooxy-tris-pyrrolidino-phosphonium hexafluorophosphate (PyOxim); O-[(ethoxycarbonyl) cyanomethylenamino]-N,N,N′,N′-tetramethyluronium tetrafluoroborate (TOTU); N,N,N′,N′-tetramethyl-O—(N-succinimidyl) uronium tetrafluoroborate (TSTU); or 1,1,3,3-tetramethyl-2-(4,5,6,7-tetrachloro-1,3-dioxoisoindolin-2-yl) isouronium hexafluorophosphate (V) (CITU).

Chiral amino acid conjugated dextrorphan compounds of the present technology can be prepared through an esterification reaction between enantiomerically pure amino acids and dextrorphan. In certain aspects of the present technology, the synthesis can be modified with additional chemicals that preserve the enantiomeric purity of the chiral amino acid during the esterification reaction.

To prepare N-acetyl-amino acid prodrugs of dextrorphan, N-acetyl-L-amino acids were reacted directly with dextrorphan as outlined in Scheme 1. Despite using the same reaction conditions for all amino acids, partial conversion of Ac-L-Val-OH (about 50%) and Ac-L-Ile-OH (about 75%) to the respective D-isomer was observed in the dextrorphan prodrug products. However, no chiral inversion was found with Ac-L-Ala-OH, Ac-L-Phe-OH, Ac-L-Lys(Ac)—OH, and Ac-L-Glu(′Bu)-OH. Notably, the reaction times for Ac-Val-OH and Ac-Ile-OH were significantly longer compared to the other N-acetyl-L-amino acids (Table 1). Without wishing to be bound by theory, a potential explanation could be that the B-carbon in valine and isoleucine is disubstituted while the β-carbon in alanine, phenylalanine, lysine, and glutamic acid is monosubstituted.

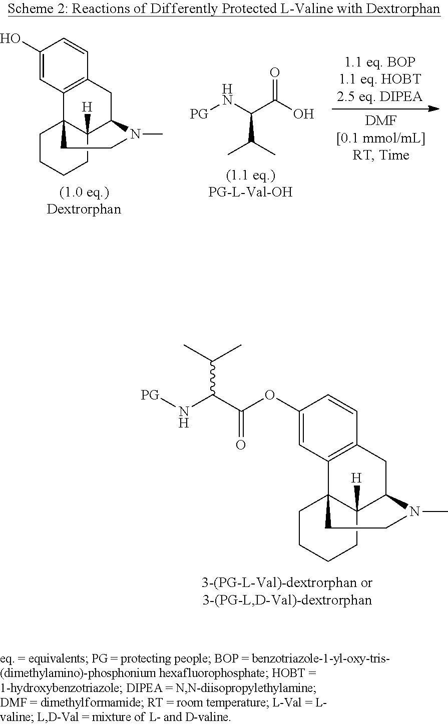

The effect of different protecting groups on the chiral inversion of L-valine were evaluated during the synthesis of valine prodrugs of dextrorphan (Scheme 2). The protecting groups acetyl, benzoyl, and phthaloyl showed about 50% chiral conversion of L-valine to D-valine in the dextrorphan prodrug product. However, no chiral inversion was observed with Boc-L-Val-OH (Table 2). While the reaction times varied significantly depending on the protecting group attached to L-valine, there was no apparent pattern with respect to the rate of chiral inversion. One potential explanation could be that the Boc-group is attached to L-valine via a carbamate bond while the three other protecting groups are bound to L-valine via an amide bond.

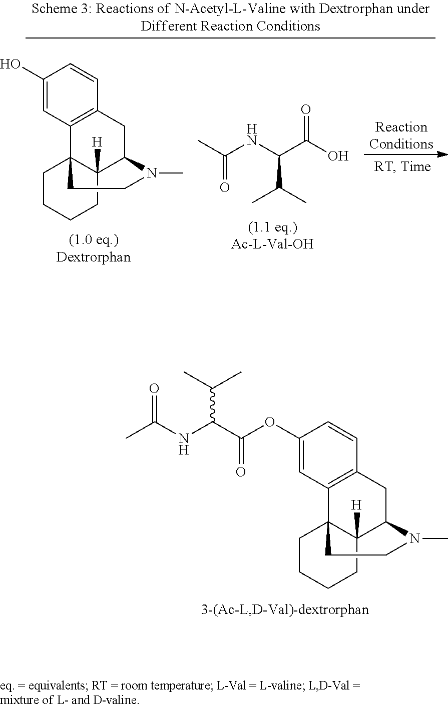

The effects of different reaction conditions and activating agents on the chiral inversion of N-acetyl-L-valine were evaluated during the synthesis of dextrorphan ester prodrugs (Scheme 3). Regardless of the activating agent and solvent, all products showed partial chiral inversion of N-acetyl-L-valine to N-acetyl-D-valine in the dextrorphan prodrug product (Table 3). No conditions were found for directly attaching N-acetyl-L-valine to dextrorphan in one step that resulted in diastereomerically pure 3-(Ac-L-Val)-dextrorphan.

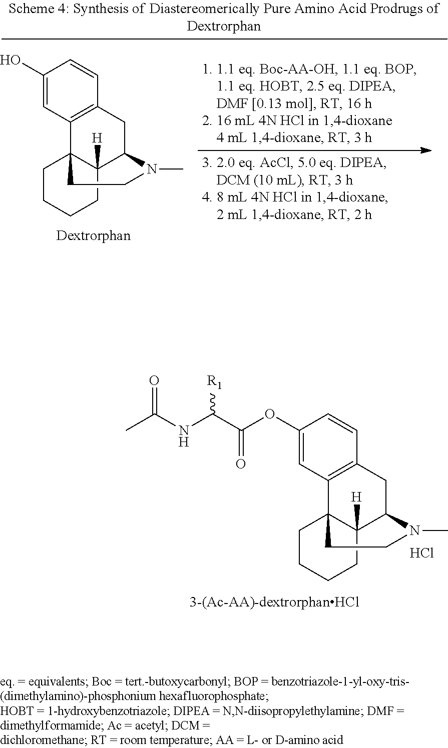

As no reaction condition could be identified for certain N-acetyl-L-amino acids or N-acetyl-D-amino acids that would produce diastereomerically pure 3-(Ac-AA)-dextrorphan (AA=L- or D-amino acid) in a one-step reaction, a multistep synthesis was developed. Experiments had shown that Boc-protected amino acids are not prone to chiral inversion during the synthesis of dextrorphan ester prodrugs. Therefore, Boc-AA-OH was reacted with dextrorphan to produce diastereomerically pure 3-(Boc-AA)-dextrorphan. Subsequent deprotection and acetylation resulted in the respective diastereomerically pure 3-(Ac-AA)-dextrorphan.

### General Procedure:

Dextrorphan freebase (400 mg, 1.55 mmol, 1.0 eq.), Boc-AA-OH (1.71 mmol, 1.1 eq.), BOP (722 mg, 1.63 mmol, 1.05 eq.), and HOBT (220 mg, 1.63 mmol, 1.05 eq.) were weighed directly into an 8-dram vial fitted with a magnetic stir bar and flushed with argon. Anhydrous DMF (12 mL) was added to the mixture to make it a homogeneous solution. To the resulting solution, DIPEA (0.68 mL, 3.88 mmol, 2.5 eq.) was added dropwise at room temperature. The reaction mixture was flushed with argon and allowed to stir at room temperature for 16 h.

DMF was removed from the reaction mixture under vacuum. The resulting light pink semi solid was dissolved in ethyl acetate (30 mL) and the organic layer was sequentially washed with 5% aq. NaHCO₃ solution (10 mL×2), water (10 mL×1), and brine solution (10 mL×1). The organic layer was dried over anhydrous Na₂SO₄ and concentrated under reduced pressure to afford sticky white solid as crude product. The crude material was purified by preparative-HPLC. Fractions containing pure product were combined and concentrated under reduced pressure. Residual water was removed by co-distillation with 3:1 toluene and acetonitrile mixture (30 mL×3) to afford diastereomerically pure Boc-amino acid ester intermediate Boc-AA-dextrorphan.

The isolated Boc-amino acid ester intermediate was dissolved in 4 mL of 1,4-dioxane and to the resulting solution was added 4N HCl in 1,4-dioxane (16 mL) at room temperature under argon atmosphere. The reaction mixture was stirred for 3 h and solvent was removed under reduced pressure. Residual HCl was removed by co-distillation with IPAc (10 mL×3) to obtain the desired deprotected amino acid ester intermediate.

The deprotected amino acid ester (0.82 mmol, 1.0 eq.) was suspended in DCM (10 mL). DIPEA (4.1 mmol, 5.0 eq.) was added to the suspension at 0-5° C. under argon atmosphere. The reaction mixture turned homogeneous after DIPEA addition. After 15 min., acetyl chloride (1.64 mmol, 2.0 eq.) was added at 0-5° C., and the mixture was gradually warmed to room temperature and stirred for 3 h.

The reaction mixture was diluted with DCM (15 mL) and the organic phase was washed with 5% aq. NaHCO₃ solution (10 mL×2). The organic layer was dried over anhydrous Na₂SO₄ and concentrated under reduced pressure to afford a sticky light-yellow solid as crude product. The crude material was purified by preparative-HPLC. Fractions containing pure product were combined and concentrated under reduced pressure. Residual water was removed by co-distillation with 3:1 toluene and acetonitrile mixture (30 mL×3) to afford diastereomerically pure 3-(Ac-AA)-dextrorphan.

The isolated 3-(Ac-AA)-dextrorphan intermediate was dissolved in 2 mL of 1,4-dioxane and to the resulting solution was added 4N HCl in 1,4-dioxane (8 mL) at room temperature under argon atmosphere. The reaction mixture was stirred for 3 h and solvent was removed under reduced pressure. Residual HCl was removed by co-distillation with IPAc (10 mL×3) to obtain 3-(Ac-AA)-dextrorphan as HCl salt.

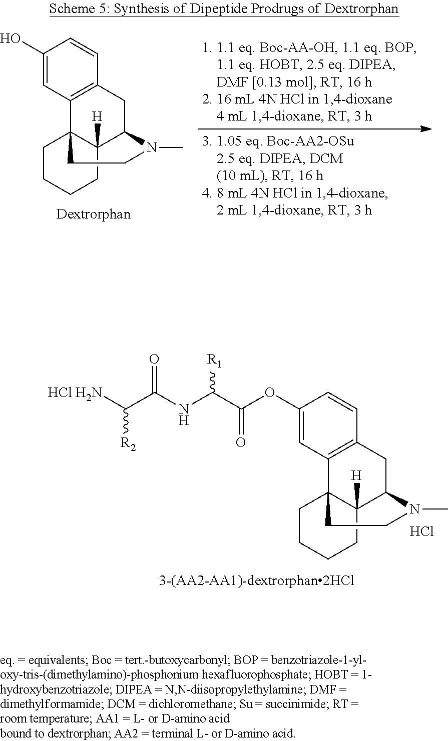

### General Procedure:

Dextrorphan freebase (400 mg, 1.55 mmol, 1.0 eq.), Boc-AA1-OH (AA1=L- or D-amino acid; 1.71 mmol, 1.1 eq.), BOP (722 mg, 1.63 mmol, 1.05 equiv), and HOBT (220 mg, 1.63 mmol, 1.05 eq.) were weighed directly into an 8-dram vial fitted with a magnetic stir bar and flushed with argon. Anhydrous DMF (12 mL) was added to the mixture to make it a homogeneous solution. To the resulting solution, DIPEA (0.68 mL, 3.88 mmol, 2.5 eq.) was added dropwise at room temperature. The reaction mixture was flushed with argon and allowed to stir at room temperature for 16 h.

DMF was removed from the reaction mixture under vacuum. The resulting light pink semi solid was dissolved in ethyl acetate (30 mL) and the organic layer was washed sequentially with 5% aq. NaHCO₃ solution (10 mL×2), water (10 mL×1), and brine solution (10 mL×1). The organic layer was dried over anhydrous Na₂SO₄ and was concentrated under reduced pressure to afford a sticky white solid as crude product. The crude material was purified by preparative-HPLC. Fractions containing pure product were combined and concentrated under reduced pressure. The residual water was removed by co-distillation with 3:1 toluene and acetonitrile mixture (30 mL×3) to afford diastereomerically pure Boc-amino acid ester 3-(Boc-AA1)-dextrorphan.

The isolated Boc-amino acid ester was dissolved in 4 mL of 1,4-dioxane and to the resulting solution was added 4N HCl in 1,4-dioxane (16 mL) at room temperature under argon atmosphere. The reaction mixture was stirred for 3 h and solvents were removed under vacuum. Residual HCl was removed by co-distillation with IPAc (10 mL×3) to obtain the desired deprotected amino acid ester intermediate 3-AA1-dextrorphan·2 HCl.

The deprotected intermediate 3-AA1-dextrorphan·2 HCl (0.82 mmol, 1.0 eq.) was suspended in DCM (10 mL). DIPEA (2.1 mmol, 2.5 eq.) was added to the suspension at room temperature under argon atmosphere. The reaction mixture turned homogeneous after DIPEA addition. After 15 min., Boc-AA2-OSu (AA2=L- or D-amino acid; 0.9 mmol, 1.05 eq.) was added to the reaction mixture at room temperature and the resulting mixture was stirred overnight.

The reaction mixture was diluted with DCM (15 mL) and the organic phase was washed with 5% aq. NaHCO₃ solution (10 mL×2). The organic layer was dried over anhydrous Na₂SO₄ and was concentrated under reduced pressure to afford a sticky light-yellow solid as crude product. The crude material was purified by preparative-HPLC. The purities of the isolated fractions were determined by HPLC. Fractions containing pure product were combined and concentrated under reduced pressure. Residual water was removed by co-distillation with 3:1 toluene and acetonitrile mixture (30 mL×3) to afford diastereomerically pure 3-(Boc-AA2-AA1)-dextrorphan.

The isolated 3-(Boc-AA2-AA1)-dextrorphan intermediate was dissolved in 2 mL of 1,4-dioxane and to the resulting solution was added 4N HCl in 1,4-dioxane (8 mL) at room temperature under argon atmosphere to remove the Boc-group. The mixture was stirred for 3 h and solvent was removed under reduced pressure. Residual HCl was removed by co-distillation with IPAc (10 mL×3) to yield the desired dipeptide 3-(AA2-AA1)-dextrorphan·2 HCl.

The chemical structures of several non-limiting chiral amino acid dextrorphan conjugates are shown in Table 1 below:

TABLE 1
Exemplary chiral amino acid dextrorphan conjugate structures.

| Compound No. | Compound Structure | Compound Name |
|---|---|---|
| 1a | 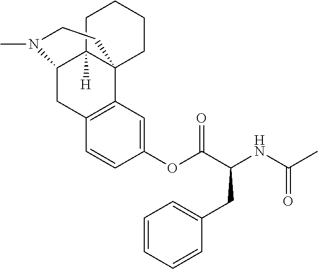 [SMILES: CC(=O)N[C@@H](Cc1ccccc1)C(=O)Oc1ccc2c(c1)[C@]13CCCC[C@@H]1[C@H](C2)N(C)CC3] | 3-(N-Ac-L-Phe)- dextrorphan |
| 1b | 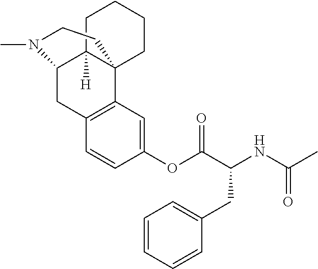 [SMILES: CC(=O)N[C@H](Cc1ccccc1)C(=O)Oc1ccc2c(c1)[C@]13CCCC[C@@H]1[C@H](C2)N(C)CC3] | 3-(N-Ac-D-Phe)- dextrorphan |
| 2a | 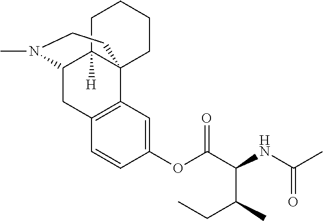 | 3-(N-Ac-L-Ile)- dextrorphan |
| 2b | 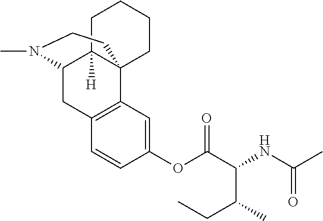 | 3-(N-Ac-D-Ile)- dextrorphan |
| 3a | 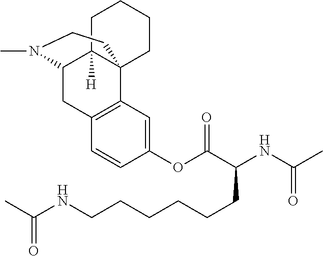 | 3-(N-Ac-L-Lys(Ac))- dextrorphan |
| 3b | 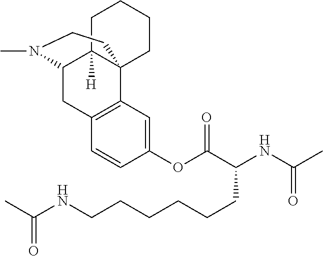 | 3-(N-Ac-D-Lys(Ac))- dextrorphan |
| 4a | 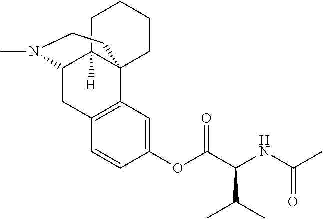 | 3-(N-Ac-L-Val)- dextrorphan |
| 4b | 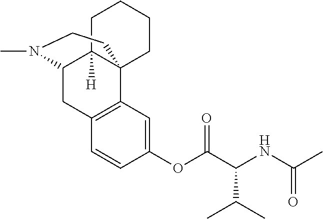 | 3-(N-Ac-D-Val)- dextrorphan |
| 5a | 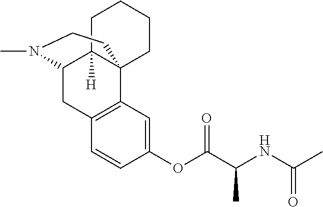 | 3-(N-Ac-L-Ala)- dextrorphan |
| 5b | 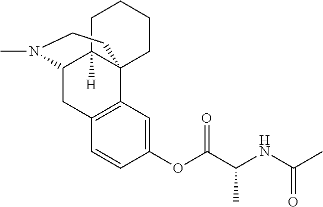 | 3-(N-Ac-D-Ala)- dextrorphan |
| 6a | 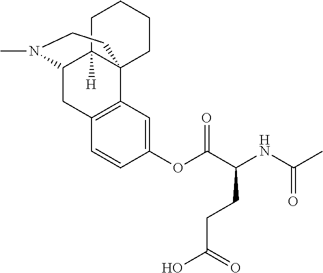 | 3-(N-Ac-L-Glu)- dextrorphan |
| 6b | 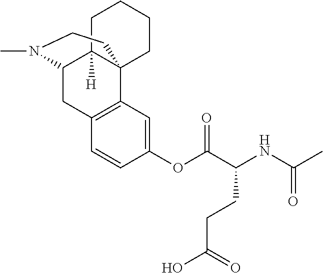 | 3-(N-Ac-D-Glu)- dextrorphan |
| 7a | 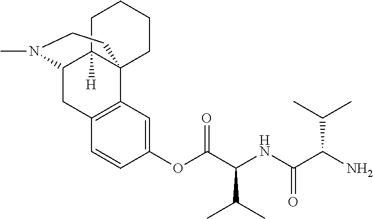 | 3-(L-Val-L-Val)- dextrorphan |
| 7b | 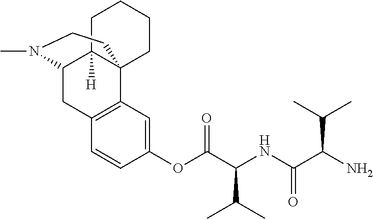 | 3-(D-Val-D-Val)- dextrorphan |
| 8a | 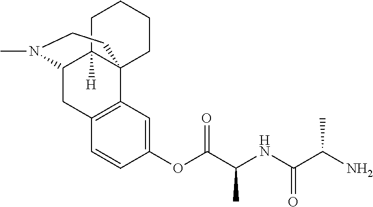 | 3-(L-Ala-L-Ala)- dextrorphan |
| 8b | 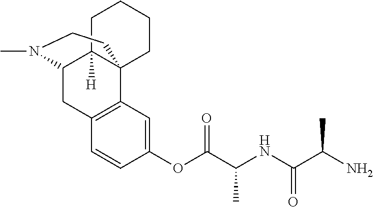 | 3-(D-Ala-D-Ala)- dextrorphan |
| 9 | 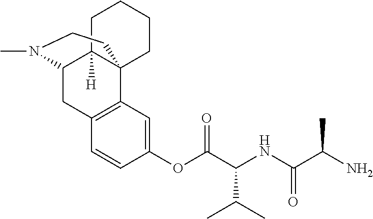 | 3-(D-Ala-D-Val)- dextrorphan |

In an embodiment, the present disclosure provides a method of manufacturing a compound having a structure of Formula II:

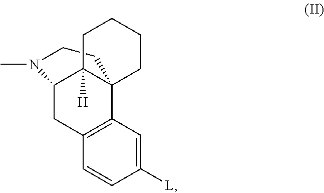

where L is A¹-G¹, A¹ is an optically active amino acids, wherein the optically active amino acid has a chiral center, and G¹ is an acyl group, and the method comprising: reacting a protected diestereomerically pure amino acid with dextrorphan in a solvent in the presence of a base for form a protected amino acid ester intermediate; and reacting the protected amino acid ester intermediate with an acid to produce a compound of Formula II.

In an embodiment, the present disclosure provides a method of manufacturing a compound having a structure of Formula II, wherein the protected disetereomerically pure amino acid is protected by a protecting group selected from the group consisting of acetyl (Ac), tert-butyoxycarbonyl (Boc), tert-butyl (′Bu), benzyloxycarbonyl (Cbz), p-methoxybenzylcarbonyl (Moz), 9-fluorenylmethyloxycarbonyl (Fmoc), benzyl (Bn), benzoyl (Bz), phthaloyl, p-methoxybenzyl (PMB), 3,4 dimethoxybenzyl (DMPM), p-methozyphenyl (PMP), tosyl (Ts), acetamide, and pthalamide.

In an embodiment, the present disclosure provides a method of manufacturing a compound having a structure of Formula II, wherein the protecting group is tert-butyoxycarbonyl (Boc).

In an embodiment, the present disclosure provides a method of manufacturing a compound having a structure of Formula II, wherein the base is selected from the group consisting of 4-methylmorpholine (NMM), 4-(dimethylamino)pyridine (DMAP), N,N-diisopropylethylamine (DIPEA), lithium bis(trimethylsilyl)amide, lithium diisopropylamide (LDA), potassium tert.-butoxide, sodium tert-butoxide, lithium tert-butoxide, sodium hydride, potassium hydride, lithium hydride, an alkoxide, and a tertiary amine.

In an embodiment, the present disclosure provides a method of manufacturing a compound having a structure of Formula II, wherein the solvent is selected from the group consisting of acetone, acetonitrile, butanol, chloroform, dichloromethane (DCM), dimethylformamide (DMF), dimethylsulfoxide (DMSO), dioxane, ethanol, ethyl acetate, diethyl ether, heptane, hexane, methanol, methyl tert.-butyl ether (MTBE), isopropanol, isopropyl acetate, diisopropyl ether, tetrahydrofuran, toluene, xylene, and water.

In an embodiment, the present disclosure provides a method of manufacturing a compound having a structure of Formula III:

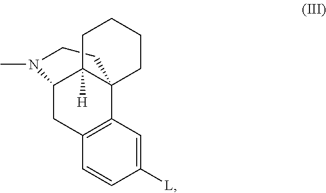

where L is A¹-A²-G², A¹ and A² are independently selected optically active amino acids, wherein at least one of A¹ and A² has a chiral center, and G² is an acyl group or absent; the method comprising: reacting a first protected diestereomerically pure amino acid with dextrorphan in a solvent in the presence of a base to produce a first intermediate; reacting the first intermediate with an acid to form a deprotected amino acid ester intermediate; reacting the deprotected amino acid ester intermediate with a second protected diestereomerically pure amino acid to produce a protected dipeptide intermediate in a second solvent in the presence of a second base; and reacting the protected dipeptide intermediate with an acid to produce a compound of Formula III.

In an embodiment, the present disclosure provides a method of manufacturing a compound having a structure of Formula III, wherein the protected disetereomerically pure amino acid is protected by a first protecting group selected from the group consisting of acetyl (Ac), tert-butyoxycarbonyl (Boc), tert-butyl (′Bu), benzyloxycarbonyl (Cbz), p-methoxybenzylcarbonyl (Moz), 9-fluorenylmethyloxycarbonyl (Fmoc), benzyl (Bn), benzoyl (Bz), phthaloyl, p-methoxybenzyl (PMB), 3,4 dimethoxybenzyl (DMPM), p-methozyphenyl (PMP), tosyl (Ts), acetamide, and pthalamide. Alternatively, A¹ and A² are independently selected amino acids, wherein at least one of A¹ and A² has a chiral center and is optically active.

In an embodiment, the present disclosure provides a method of manufacturing a compound having a structure of Formula III, wherein the protecting group is tert-butyoxycarbonyl (Boc).

In an embodiment, the present disclosure provides a method of manufacturing a compound having a structure of Formula III, wherein the base is selected from the group consisting of 4-methylmorpholine (NMM), 4-(dimethylamino)pyridine (DMAP), N,N-diisopropylethylamine (DIPEA), lithium bis(trimethylsilyl)amide, lithium diisopropylamide (LDA), potassium tert.-butoxide, sodium tert-butoxide, lithium tert-butoxide, sodium hydride, potassium hydride, lithium hydride, an alkoxide, and a tertiary amine.

In an embodiment, the present disclosure provides a method of manufacturing a compound having a structure of Formula III, wherein the solvent is selected from the group consisting of acetone, acetonitrile, butanol, chloroform, dichloromethane (DCM), dimethylformamide (DMF), dimethylsulfoxide (DMSO), dioxane, ethanol, ethyl acetate, diethyl ether, heptane, hexane, methanol, methyl tert.-butyl ether (MTBE), isopropanol, isopropyl acetate, diisopropyl ether, tetrahydrofuran, toluene, xylene, and water.

The figures show oral pharmacokinetic (PK) data for several non-limiting aspects of the present technology. Area Under the Curve (AUC), maximum plasma concentration (Cₘₐₓ) and time to maximum plasma concentration Tₘₐₓ data is described as follows:

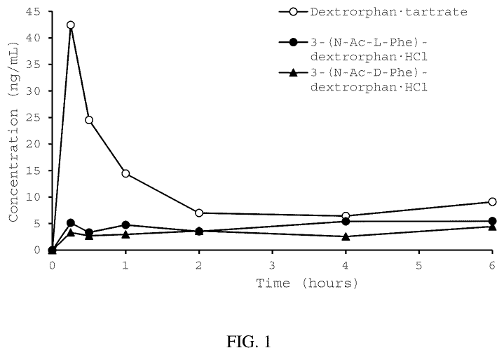

FIG. 1 compares the oral PK profiles of dextrorphan tartrate and the L- and D-Phe diastereomers of 3-(N—Ac-Phe)-dextrorphan HCl (1a and 1b). Both the 3-(N—Ac-L-Phe)-dextrorphan HCl and 3-(N—Ac-D-Phe)-dextrorphan HCl have a much smaller AUC and Cₘₐₓ values compared to dextrorphan tartrate. The prodrugs also show a much steadier concentration over the 6 hour observation time, compared to dextrorphan tartrate which has a rapid rise to Cₘₐₓ followed by a substantial drop in plasma concentration.

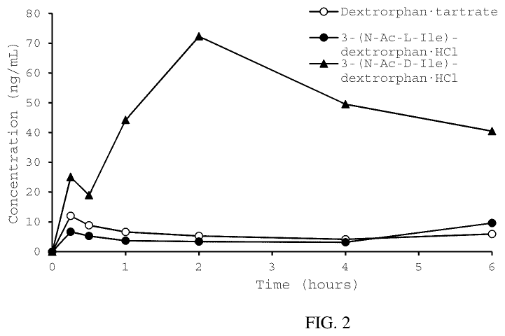

FIG. 2 compares the oral PK profiled of dextrorphan tartrate and the L- and D-Ile diastereomers of 3-(N—Ac-Ile)-dextrorphan HCl (2a and 2b). Both the 3-(N—Ac-L-Ile)-dextrorphan HCl and 3-(N—Ac-D-Ile)-dextrorphan HCl have a much smaller AUC and Cₘₐₓ values compared to dextrorphan tartrate.

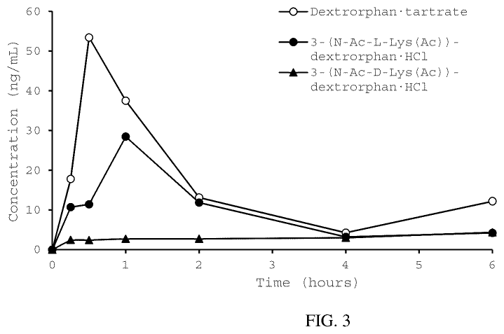

FIG. 3 compares the oral PK profiles of dextrorphan tartrate and the L- and D-Lys diastereomers of 3-(N—Ac-Lys(Ac))-dextrorphan HCl (3a and 3b). Both the 3-(N—Ac-L-Lys(Ac))-dextrorphan HCl and 3-(N—Ac-D-Lys(Ac))-dextrorphan HCl have a much smaller AUC and Cₘₐₓ values compared to dextrorphan tartrate. The 3-(N—Ac-D-Lys(Ac))-dextrorphan HCl shows a lower and steadier blood concentration than 3-(N—Ac-L-Lys(Ac))-dextrorphan HCl.

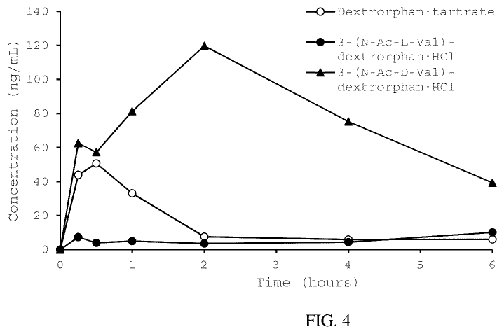

FIG. 4 compares the oral PK profiles of dextrorphan tartrate and the L- and D-Val diastereomers of 3-(N—Ac-Val)-dextrorphan HCl (4a and 4b). The 3-(N—Ac-D-Val)-dextrorphan HCl shows a higher AUC and Cₘₐₓ compared to dextrorphan tartrate and 3-(N—Ac-L-Val)-dextrorphan HCl. The 3-(N—Ac-L-Val)-dextrorphan HCl shows a lower AUC and Cₘₐₓ than dextrorphan tartrate.

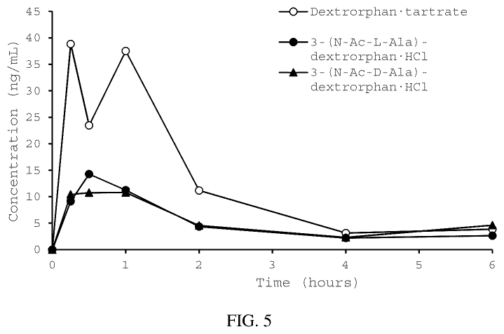

FIG. 5 compares the oral PK profiles of dextrorphan tartrate and the L- and D-Ala diastereomers of 3-(N—Ac-Ala)-dextrorphan HCl (5a and 5b). Both the 3-(N—Ac-L-Ala)-dextrorphan HCl and 3-(N—Ac-D-Ala)-dextrorphan HCl have a much smaller AUC and Cₘₐₓ value compared to dextrorphan tartrate. Both prodrugs also show a steadier plasma concentration compared to dextrorphan tartrate.

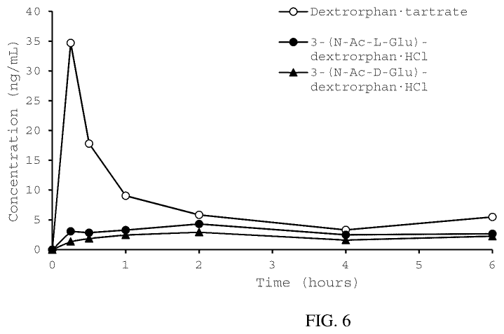

FIG. 6 compares the oral PK profiles of dextrorphan tartrate and the L- and D-Glu diastereomer of 3-(N—Ac-L-Glu)-dextrorphan HCl (6a and 6b). Both the 3-(N—Ac-L-Glu)-dextrorphan HCl and 3-(N—Ac-D-Glu)-dextrorphan HCl have a much smaller AUC and Cₘₐₓ value compared to dextrorphan tartrate. Both prodrugs also show a steadier plasma concentration compared to dextrorphan tartrate and reach their maximum plasma concentration around 2 hours after administration compared to approximately 30 minutes after administration of dextrorphan tartrate.

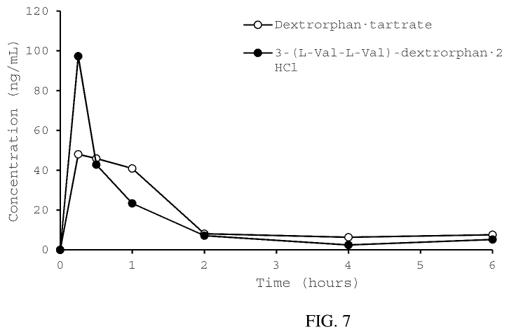

FIG. 7 compares the oral PK profiles of dextrorphan tartrate and 3-(L-Val-L-Val)-dextrorphan 2 HCl (7a). The prodrug has a larger Cₘₐₓ compared to dextrorphan tartrate.

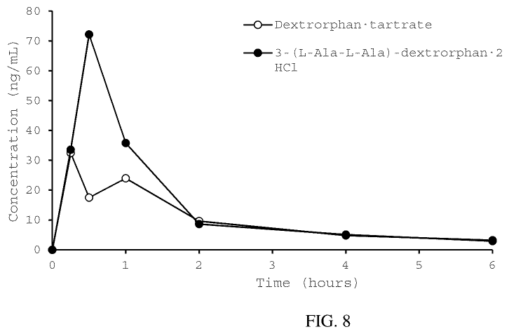

FIG. 8 compares the oral PK profiles of dextrorphan tartrate and 3-(L-Ala-L-Ala)-dextrorphan 2 HCl (8a). The prodrug has a larger Cₘₐₓ and a larger AUC compared to dextrorphan tartrate.

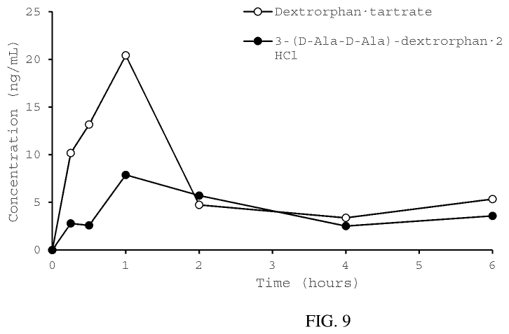

FIG. 9 compares the oral PK profiles of dextrorphan tartrate and 3-(D-Ala-D-Ala)-dextrorphan 2 HCl (8b). The prodrug has a smaller Cₘₐₓ and a lower AUC than dextrorphan tartrate.

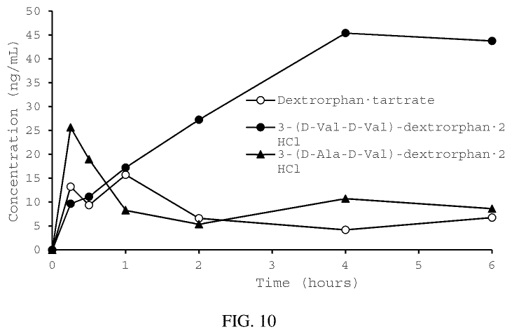

FIG. 10 compares the oral PK profiles of dextrorphan tartrate, 3-(D-Val-D-Val)-dextrorphan 2 HCl (7b), and 3-(D-Ala-D-Val)-dextrorphan 2 HCl (9a). Both prodrugs have a larger Cₘₐₓ and a larger AUC than dextrorphan tartrate, and 3-(D-Val-D-Val)-dextrorphan 2 HCl has a larger Cₘₐₓ and a larger AUC compared to 3-(D-Ala-D-Val)-dextrorphan 2 HCl. The prodrug has a smaller Cₘₐₓ and a lower AUC than dextrorphan tartrate.

### Pharmaceutical Compositions Comprising Chiral Amino Acid Conjugates of Dextrorphan

In an embodiment, the present disclosure provides a composition comprising: a therapeutically effective amount of a compound having a structure of Formula I:

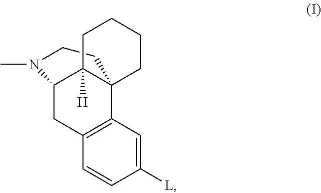

where L is A¹-G¹ or A¹-A²-G², and A¹ and A² are independently selected optically active amino acids, wherein at least one of the A¹ and/or A² has a chiral center, G¹ is an acyl group, and G² is an acyl group or absent; or a pharmaceutically acceptable salt thereof. Alternatively, A¹ and A² are independently selected amino acids, wherein at least one of A¹ and A² has a chiral center and is optically active.

In some embodiments, the present disclosure describes at least one prodrug composition comprising at least one conjugate as described above. The at least one conjugate may comprise at least one dextrorphan and at least one amino acid and/or acyl amino acid, or derivatives thereof, or salts thereof. The at least one amino acid and/or acyl amino acid may comprise at least one L-isomer or(S)-configuration at the α-carbon of the amino acid or acyl amino acid, or at least one D-isomer or (R)-configuration at the α-carbon of the amino acid or acyl amino acid, or combinations thereof.

Depending on the at least one isomer of the amino acid and/or acyl amino acid, conjugated to dextrorphan or derivative thereof, the at least one prodrug formed can be either a neutral (uncharged), a free acid, a free base or a pharmaceutically acceptable anionic salt form or salt mixtures with any ratio between positive and negative components. These anionic salt forms can include, but are not limited to, for example, acetate, l-aspartate, besylate, bicarbonate, carbonate, d-camsylate, l-camsylate, citrate, edisylate, formate, fumarate, gluconate, hydrobromide/bromide, hydrochloride/chloride, d-lactate, l-lactate, d,l-lactate, d,l-malate, l-malate, mesylate, pamoate, phosphate, succinate, sulfate, bisulfate, d-tartrate, l-tartrate, d,l-tartrate, meso-tartrate, benzoate, gluceptate, d-glucuronate, hybenzate, isethionate, malonate, methylsulfate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate (mucate), galacturonate, gallate, gentisate, glutamate, glutamate, glutarate, glycerophosphate, heptanoate (enanthate), hydroxybenzoate, hippurate, phenylpropionate, iodide, xinafoate, lactobionate, laurate, maleate, mandelate, methanesulfonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, or undecylenate.

In some embodiments, the composition comprising the at least one conjugate of dextrorphan, the composition further comprises at least one excipients selected from antiadherents, binders, coatings, disintegrants, gel forming agents, fillers, flavors and colors, glidants, lubricants, preservatives, sorbents and sweeteners, and combinations thereof.

In some embodiments, the composition comprising the at least one conjugate of dextrorphan, the composition is a oral dosage formulation. “Oral dosage formulation,” as it relates to the present technology mean formulations that include but are not limited to tablet, capsule, caplet, troche, lozenge, powder, suspension, syrup, solution, oral thin film (OTF), oral strips, and inhalation compounds. Preferred oral administration forms are solutions, syrups, suspensions, capsules, tablets and OTF. Suitable dosing vehicles of the present technology include, but are not limited to, water, phosphate buffered saline (PBS), Tween in water, and PEG in water.

In some embodiments, the oral dosage formulation is a solid dosage formulation. Solid dosage forms can optionally include one or more of the following types of excipients: antiadherents, binders, coatings, disintegrants, gel-forming agents, fillers, flavors and colors, glidants, lubricants, preservatives, sorbents and sweeteners, among others.

Oral formulations of the present technology can also be included in a solution, a suspension or a slurry, in an aqueous liquid or a non-aqueous liquid. The formulation can be an emulsion, such as an oil-in-water liquid emulsion or a water-in-oil liquid emulsion. The oils can be administered by adding the purified and sterilized liquids to a prepared enteral formula, which is then placed in the feeding tube of a patient who is unable to swallow.

Soft gel or soft gelatin capsules may be prepared, for example by dispersing the formulation in an appropriate vehicle (vegetable oils are commonly used) to form a high viscosity mixture. This mixture is then encapsulated with a gelatin based film using technology and machinery known to those in the soft gel industry. The individual units so formed are then dried to constant weight.

Chewable tablets, for example, may be prepared by mixing the formulations with excipients designed to form a relatively soft, flavored, tablet dosage form that is intended to be chewed rather than swallowed. Conventional tablet machinery and procedures, for example, direct compression and granulation, i.e., or slugging, before compression, can be utilized. Those individuals involved in pharmaceutical solid dosage form production are versed in the processes and the machinery used, as the chewable dosage form is a very common dosage form in the pharmaceutical industry.

Film coated tablets, for example may be prepared by coating tablets using techniques such as rotating pan coating methods or air suspension methods to deposit a contiguous film layer on a tablet.

Compressed tablets, for example may be prepared by mixing the formulation with one or more excipients intended to add binding qualities to disintegration qualities. The mixture is either directly compressed, or granulated and then compressed using methods and machinery known to those in the industry. The resultant compressed tablet dosage units are then packaged according to market need, for example, in unit dose, rolls, bulk bottles, blister packs, etc.

The present technology contemplates that the conjugates of the present technology can be formulated into formulations or co-formulations that may further comprise one or more additional components. For example, such formulations can include biologically-acceptable carriers which may be prepared from a wide range of materials. Without being limited to, such materials include diluents, binders and adhesives, lubricants, gel-forming agents, plasticizers, disintegrants, surfactants, colorants, bulking substances, flavorings, sweeteners and miscellaneous materials such as buffers and adsorbents in order to prepare a particular medicated formulation.

Binders may be selected from a wide range of materials such as hydroxypropylmethylcellulose, ethylcellulose, or other suitable cellulose derivatives, povidone, acrylic and methacrylic acid co-polymers, pharmaceutical glaze, gums, milk derivatives, such as whey, starches, and derivatives, as well as other conventional binders known to persons working in the art. Exemplary non-limiting solvents are water, ethanol, isopropyl alcohol, methylene chloride or mixtures and combinations thereof. Exemplary non-limiting bulking substances include sugar, lactose, gelatin, starch, and silicon dioxide.

It should be understood that in addition to the ingredients particularly mentioned above, the formulations of the present technology can include other suitable agents such as flavoring agents, preservatives and antioxidants. Such antioxidants would be food acceptable and could include vitamin E, carotene, BHT or other antioxidants.

Other compounds which may be included by admixture are, for example, medically inert ingredients, e.g., solid and liquid diluents, such as lactose, dextrose, saccharose, cellulose, starch or calcium phosphate for tablets or capsules, olive oil or ethyl oleate for soft capsules and water or vegetable oil for suspensions or emulsions; lubricating agents such as silica, talc, stearic acid, magnesium or calcium stearate and/or polyethylene glycols; gelling agents such as colloidal clays; thickening agents such as gum tragacanth or sodium alginate, binding agents such as starches, arabic gums, gelatin, methylcellulose, carboxymethylcellulose or polyvinylpyrrolidone; disintegrating agents such as starch, alginic acid, alginates or sodium starch glycolate; effervescing mixtures; dyestuff; sweeteners; wetting agents such as lecithin, polysorbates or laurylsulfates; and other therapeutically acceptable accessory ingredients, such as humectants, preservatives, buffers and antioxidants, which are known additives for such formulations.

For oral administration, fine powders or granules containing diluting, dispersing and/or surface-active agents may be presented in a draught, in water or a syrup, in capsules or sachets in the dry state, in a non-aqueous suspension wherein suspending agents may be included, or in a suspension in water or a syrup. Where desirable, flavoring, preserving, suspending, thickening or emulsifying agents can be included.

Liquid dispersions for oral administration may be syrups, emulsions or suspensions. The syrups may contain as carrier, for example, saccharose or saccharose with glycerol and/or mannitol and/or sorbitol. In particular, a syrup for diabetic patients can contain as carriers only products, for example sorbitol, which do not metabolize to glucose or which metabolize only a very small amount to glucose. The suspensions and the emulsions may contain a carrier, for example a natural gum, agar, sodium alginate, pectin, methylcellulose, carboxymethylcellulose or polyvinyl alcohol. The ingredients mentioned herein are not intended to be exhaustive, and one of skill in the art will be able to formulate compositions using known or to be known ingredients.

In a still further embodiment, the present disclosure describes a composition comprising therapeutically effective amount of a chiral amino acid ester prodrug of dextrorphan, wherein the composition is a solid oral dosage formulation in the form of a unit dosage.

“Unit dose form,” or “unit dosage,” as it relates to the present technology means a single entity of a solid therapeutic dosage form (e.g., 1 capsule, 1 tablet) or a single volume dispensed from a non-solid dosage form (e.g., 5 mL of a liquid or syrup). Such a unit dose form can be from about 0.1 mg to about 500 mg per day, alternatively from about 0.1 mg to about 400 mg per day, about 0.1 mg to about 300 mg per day, alternatively about 1 mg to about 100 mg per day, alternatively about 5 mg to about 80 mg per day, alternatively about 10 mg to about 40 mg per day, alternatively about 10 mg to 200 mg per day, alternatively about 20 mg to about 120 mg per day, alternatively about 30 mg to about 100 mg per day, alternatively about 40 mg to about 80 mg per day, alternatively about 50 mg to about 70 mg per day, alternatively about 20 mg to about 40 mg per day, alternatively about 20 mg to about 60 mg per day, alternatively about 10 mg to about 50 mg per day, alternatively about 20 mg per day, alternatively about 40 mg per day, alternatively about 60 mg per day, alternatively 80 mg per day, alternatively 100 mg per day, alternatively 160 mg per day, alternatively 240 mg per day, alternatively 320 mg per day.

It is contemplated that the dextrorphan conjugates of the present technology can be combined with one or more active substances, such as different dextrorphan conjugates, unconjugated dextrorphan, or other active ingredient(s) depending on intended indication. Examples of active pharmaceuticals that can be combined with the conjugates of the present technology include, but are not limited to, acetaminophen, phenylpropanolamine, ibuprofen, aspirin, diflusinal, salicylic acid, dexibuprofen, naproxen, fenoprofen, ketoprofen, dexketoprofen, flurbiprofen, oxaprozin, loxoprofen, indomethacin, tolmetin, sulindac, etodolac, ketorolac, diclofenac, piroxicam, meloxicam, tenoxicam, lornoxicam, isoxicam, mefenamic acid, meclofenamic acid, flufenamic acid, tolfenamic acid, celecoxib, valdecoxib, lumiracoxib, pheniramine, chlorpheniramine, fexofenadine, azelastine, hydroxyzine, diphenhydramine, desloratidine, loratidine, cyproheptadine, bromopheniramine, emedastine, levocabastine, carbinoxamine, levocetirizine, clemastine, cetirizine, phenylephrine, pseudoephedrine, oxymetazoline, pyrilamine, doxylamine, codeine, pholcodine, dextromethorphan, noscapine, butamirate, acetylcysteine, menthol, quetiapine, and guaifenesin. The conjugated dextrorphan of the present technology can be formulated with one or a combination of these or other active substances, or as a stand alone active ingredient without any other actives.

The amounts and relative percentages of the different active and inactive components of the formulations of the current technology can be modified, selected and adjusted in order to arrive at desirable formulations, dosages and dosage forms for therapeutic administration of the compounds, products, compositions, conjugates and prodrugs of the current technology.

In a further embodiment, the present disclosure provides a composition comprising: a therapeutically effective amount of a compound having a structure of Formula I, where the chiral amino acid is a standard amino acid, a non-standard amino acid, or a synthetic amino acid. Alternatively, the chiral amino acid is a standard amino acid.

In a further embodiment, the present disclosure provides a composition comprising: a therapeutically effective amount of a compound having a structure of Formula I, wherein the standard amino acid is selected from the group consisting of isoleucine, valine, or alanine. In a still aspect, the standard amino acid is selected from D-isoleucine, D-valine, and L-alanine.

In a further embodiment, the present disclosure provides a composition comprising: a therapeutically effective amount of a compound having a structure of Formula I, wherein the therapeutically effective amount of the compound of Formula I ranges from about 0.1 mg to about 2000 mg per day.

In a further embodiment, the present disclosure provides a composition comprising: a therapeutically effective amount of a compound having a structure of Formula I, wherein the composition further comprises at least one excipient selected from the group consisting of antiadherents, binders, coatings, disintegrants, gel forming agents, fillers, flavors and colors, glidants, lubricants, preservatives, sorbents and sweeteners.

In a further embodiment, the present disclosure provides a composition comprising: a therapeutically effective amount of a compound having a structure of Formula I, wherein the composition is an oral dosage formulation. In a further aspect, the oral dosage formulation is a liquid dosage formulation selected from the group consisting of syrups, emulsions or suspensions. In a still further aspect, the oral dosage formulation is a solid dosage formulation selected from the group consisting of a sublingual, a gummy, a chewable tablet, a rapidly dissolving tablet, a tablet, a capsule, a caplet, a troche, a lozenge, an oral powder, a thin strip, an oral thin film (OTF), and an oral strip

In a further embodiment, the present disclosure provides a composition comprising: a therapeutically effective amount of a compound having a structure of Formula I, wherein: the compound of Formula I has a structure of Formula II:

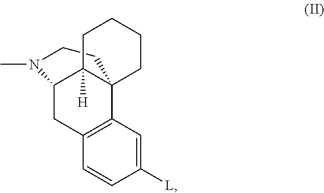

where L is A¹-G¹, and A¹ is an optically active amino acids, wherein the optically active amino acid has a chiral center, G¹ is an acyl group, or a pharmaceutically acceptable salt thereof.

In a further embodiment, the present disclosure provides a composition comprising: a therapeutically effective amount of a compound having a structure of Formula I, wherein: the compound of Formula I has a structure of Formula II, where the chiral amino acid is a standard amino acid, a non-standard amino acid, or a synthetic amino acid. In a still further aspect, the compound of Formula I has a structure of Formula II, wherein the chiral amino acid is a standard amino acid.

In a further embodiment, the present disclosure provides a composition comprising: a therapeutically effective amount of a compound having a structure of Formula I, wherein: the compound of Formula I has a structure of Formula II, wherein the standard amino acid is selected from the group consisting of isoleucine, valine, or alanine. In a further aspect, the standard amino acid is selected from D-isoleucine, D-valine, and L-alanine.

In a further embodiment, the present disclosure provides a composition comprising: a therapeutically effective amount of a compound having a structure of Formula I, wherein: the compound of Formula I has a structure of Formula II, wherein the therapeutically effective amount of the compound of Formula I ranges from about 0.1 mg to about 2000 mg per day.

In a further embodiment, the present disclosure provides a composition comprising: a therapeutically effective amount of a compound having a structure of Formula I, wherein: the compound of Formula I has a structure of Formula II, wherein the composition further comprises at least one excipient selected from the group consisting of antiadherents, binders, coatings, disintegrants, gel forming agents, fillers, flavors and colors, glidants, lubricants, preservatives, sorbents and sweeteners.

In a further embodiment, the present disclosure provides a composition comprising: a therapeutically effective amount of a compound having a structure of Formula I, wherein: the compound of Formula I has a structure of Formula II, wherein the composition is an oral dosage formulation.

In a further embodiment, the present disclosure provides a composition comprising: a therapeutically effective amount of a compound having a structure of Formula I, wherein: the compound of Formula I has a structure of Formula II, wherein the oral dosage formulation is a liquid dosage formulation selected from the group consisting of syrups, emulsions or suspensions.

In a further embodiment, the present disclosure provides a composition comprising: a therapeutically effective amount of a compound having a structure of Formula I, wherein: the compound of Formula I has a structure of Formula II, wherein the oral dosage formulation is a solid dosage formulation selected from the group consisting of a sublingual, a gummy, a chewable tablet, a rapidly dissolving tablet, a tablet, a capsule, a caplet, a troche, a lozenge, an oral powder, a thin strip, an oral thin film (OTF), and an oral strip.

In a further embodiment, the present disclosure provides a composition comprising: a therapeutically effective amount of a compound having a structure of Formula I, wherein: the compound of Formula I has a structure of Formula III:

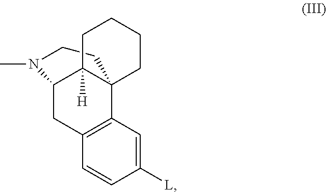

where L is A¹-A²-G², and A¹ and A² are independently selected optically active amino acids, wherein at least one of A¹ and/or A² has a chiral center, and G² is an acyl group or absent; or a pharmaceutically acceptable salt thereof. Alternatively, A¹ and A² are independently selected amino acids, wherein at least one of A¹ and A² has a chiral center and is optically active.

In a further embodiment, the present disclosure provides a composition comprising: a therapeutically effective amount of a compound having a structure of Formula I, wherein: the compound of Formula I has a structure of Formula III, wherein the at least one chiral amino acid is a standard amino acid.

In a further embodiment, the present disclosure provides a composition comprising: a therapeutically effective amount of a compound having a structure of Formula I, wherein: the compound of Formula I has a structure of Formula III, wherein A¹ is L-alanine and A² is L-alanine. In a further aspect, A¹ is D-valine and A² is D-valine.

In a further embodiment, the present disclosure provides a composition comprising: a therapeutically effective amount of a compound having a structure of Formula I, wherein: the compound of Formula I has a structure of Formula III, wherein the therapeutically effective amount of the compound of Formula I ranges from about 0.1 mg to about 2000 mg per day.

In a further embodiment, the present disclosure provides a composition comprising: a therapeutically effective amount of a compound having a structure of Formula I, wherein: the compound of Formula I has a structure of Formula III, wherein the composition further comprises at least one excipient selected from the group consisting of antiadherents, binders, coatings, disintegrants, gel forming agents, fillers, flavors and colors, glidants, lubricants, preservatives, sorbents and sweeteners.

In a further embodiment, the present disclosure provides a composition comprising: a therapeutically effective amount of a compound having a structure of Formula I, wherein: the compound of Formula I has a structure of Formula III, wherein the composition is an oral dosage formulation.

In a further embodiment, the present disclosure provides a composition comprising: a therapeutically effective amount of a compound having a structure of Formula I, wherein: the compound of Formula I has a structure of Formula III, wherein the oral dosage formulation is a liquid dosage formulation selected from the group consisting of syrups, emulsions or suspensions.

In a further embodiment, the present disclosure provides a composition comprising: a therapeutically effective amount of a compound having a structure of Formula I, wherein: the compound of Formula I has a structure of Formula III, wherein the oral dosage formulation is a solid dosage formulation selected from the group consisting of a sublingual, a gummy, a chewable tablet, a rapidly dissolving tablet, a tablet, a capsule, a caplet, a troche, a lozenge, an oral powder, a thin strip, an oral thin film (OTF), and an oral strip.

In some aspects, the present technology provides a composition comprising an ester prodrug of dextrorphan with greater bioavailability compared to unconjugated dextrorphan. In at least one aspect, the compositions/formulations of the current technology can lessen common side effects associated with unconjugated dextrorphan and similar compounds. In at least one other aspect, the compositions of the current technology can be orally administered to a human or animal patient at lower doses but with equivalent therapeutic effect compared to unconjugated dextrorphan.

In one aspect, the present technology provides a composition comprising at least one chiral amino acid ester conjugate of dextrorphan, where the at least one chiral amino acid ester conjugate comprises at least one amino acid and/or acyl amino acid, or derivatives thereof. In another embodiment the at least one amino acid and/or acyl amino acid, or derivatives thereof has a chiral center.

### Pharmaceutical Kits

In some embodiments, the present technology provides pharmaceutical kits comprising a composition of the present technology. In some embodiments, the pharmaceutical kit comprises a specific amount of individual doses in a package, each dose comprising a pharmaceutically and/or therapeutically effective amount of the composition comprising the prodrug or conjugate of the present technology. The pharmaceutical kit may further include instructions for use. In some other embodiments, the kit comprises oral thin films or strips comprising the composition comprising the prodrugs or conjugates of the present technology. In some other embodiments, the kit comprises one or more blister packs containing the composition comprising the prodrug or conjugate of the present technology. It will be appreciated by one skilled in the art that, in some embodiments, the kit may include individual doses that have different dosage amounts.

The subject may be a human or animal subject. As used herein the term animal is used in the veterinary sense and does not include humans. Suitable human subjects include neonatal subjects, pediatric subjects, adolescent subjects, adult subjects, geriatric subjects, elderly subjects and normative subjects. The kit comprises a specific amount of the individual doses in a package, each dose containing a pharmaceutically and/or therapeutically effective amount of at least one conjugate of d-methylphenidate of the present technology. The specified amount of individual doses may be from about 1 to about 100 individual dosages, alternatively from about 1 to about 60 individual dosages, alternatively from about 10 to about 30 individual dosages, including, about 1, about 2, about 5, about 10, about 15, about 20, about 25, about 30, about 35, about 40, about 45, about 50, about 55, about 60, about 70, about 80, about 100, and include any additional increments thereof, for example, about 1, about 2, about 5, about 10 and multiplied factors thereof, (e.g., about ×1, about ×2, about ×2.5, about ×5, about ×10, about ×100, etc.). One of skill in the art will appreciate that some embodiments of the kit of the present technology may include graduated individual doses (i.e. dose amounts that increase or decrease over a period of time), and/or a graduated dosing regimen, and instructions for use.

### Method of Treatment

In one embodiment, the present technology describes a method of treating a disease or disorder in a patient in need thereof, the method comprising administering to the patient in need thereof a composition comprising a therapeutically effective amount of a compound having a structure of Formula I. Alternatively, the compound of Formula I is a compound having a structure of Formula II. Alternatively, the compound of Formula I is a compound having a structure of Formula III.

In a further embodiment, the present technology describes a method of treating a disease or disorder in a patient in need thereof, the method comprising administering to the patient in need thereof a composition comprising a chiral amino acid ester conjugate of dextrorphan having a structure of Formula I, wherein the therapeutically effective amount ranges from about 0.1 mg to about 1000 mg per day.

In a further embodiment, the present technology describes a method of treating a disease or disorder in a patient in need thereof, the method comprising administering to the patient in need thereof a composition comprising a chiral amino acid ester conjugate of dextrorphan having a structure of Formula I; wherein the disease or disorder is a disease, disorder, condition, or syndrome requiring or mediated by binding of an NMDA receptor antagonist to an NMDA receptor of the patient. In some embodiments, compositions comprising the dextrorphan conjugates of the present technology may be used as an anesthetic, or for the treatment of such conditions as opioid dependence, hyperalgesia, pseudobulbar affect (PBA), neuropathy, diabetic peripheral neuropathic pain, catalepsy, amnesia, Alzheimer's disease, depression, and post-traumatic stress disorder (PTSD).

In certain embodiments, the compositions comprising the dextrorphan conjugates of the present technology may be used in combination with quinidine for the treatment of PBA and/or PTSD. In other embodiments, compositions of the present technology may potentiate the effects of certain opioids, such as for example oxycodone, in suppressing neuropathic pain, thus potentially permitting a lower dose of oxycodone to be administered to a patient and thereby decreasing side effects of oxycodone treatment of neuropathic pain in said patient.

Without wishing to be limited to the following theory, it is believed that the prodrugs/conjugates of the present technology undergo enzyme hydrolysis of the ester bond in vivo, which subsequently leads to regeneration of dextrorphan and the respective amino acid and/or acyl amino acid or metabolites thereof, and/or derivatives thereof.

### EXAMPLES

### Example 1 Synthesis of Chiral Amino Acid Conjugate Dextrorphan Prodrugs

3-(N—Ac-L-Phe)-dextrorphan HCl (1a):

Reaction of dextrorphan with Boc-L-Phe-OH is carried out in a manner similar to that described in general procedure for the synthesis of diastereomerically pure N-acetyl-amino acid prodrugs of dextrorphan (Scheme 4). The Boc-protected intermediate is deprotected with 4N HCl in dioxane to 3-L-Phe-dextrorphan 2 HCl and then acylated with acetyl chloride to give 3-(N—Ac-L-Phe)-dextrorphan that is converted to the corresponding hydrochloride salt 1a by treatment with 4N HCl in dioxane.

3-(N—Ac-D-Phe)-dextrorphan HCl (1b):

The synthesis of 3-(N—Ac-D-Phe)-dextrorphan HCl is carried out in a manner similar to that described for the synthesis of 1a.

3-(N—Ac-L-Ile)-dextrorphan HCl (2a):

The synthesis of 3-(N—Ac-L-Ile)-dextrorphan HCl is carried out in a manner similar to that described for the synthesis of 1a.

3-(N—Ac-D-Ile)-dextrorphan HCl (2b):

The synthesis of 3-(N—Ac-D-Ile)-dextrorphan HCl is carried out in a manner similar to that described for the synthesis of 1a.

3-(N—Ac-L-Lys(Ac))-dextrorphan HCl (3a):

The synthesis of 3-(N—Ac-L-Lys(Ac))-dextrorphan HCl is carried out in a manner similar to that described for the synthesis of 1a.

3-(N—Ac-D-Lys(Ac))-dextrorphan HCl (3b):

The synthesis of 3-(N—Ac-D-Lys(Ac))-dextrorphan HCl is carried out in a manner similar to that described for the synthesis of 1a.

3-(N—Ac-L-Val)-dextrorphan HCl (4a):

The synthesis of 3-(N—Ac-L-Val)-dextrorphan HCl is carried out in a manner similar to that described for the synthesis of 1a.

3-(N—Ac-D-Val)-dextrorphan HCl (4b):

The synthesis of 3-(N—Ac-D-Ile)-dextrorphan HCl is carried out in a manner similar to that described for the synthesis of 1a.

3-(N—Ac-L-Ala)-dextrorphan HCl (5a):

The synthesis of 3-(N—Ac-L-Ala)-dextrorphan HCl is carried out in a manner similar to that described for the synthesis of 1a.

3-(N—Ac-D-Ala)-dextrorphan HCl (5b):

The synthesis of 3-(N—Ac-D-Ala)-dextrorphan HCl is carried out in a manner similar to that described for the synthesis of 1a.

3-(N—Ac-L-Glu)-dextrorphan HCl (6a):

The synthesis of 3-(N—Ac-L-Glu)-dextrorphan HCl is carried out in a manner similar to that described for the synthesis of 1a.

3-(N—Ac-D-Glu)-dextrorphan HCl (6b):

The synthesis of 3-(N—Ac-D-Glu)-dextrorphan HCl is carried out in a manner similar to that described for the synthesis of 1a.

3-(L-Val-L-Val)-dextrorphan 2 HCl (7a):

Reaction of dextrorphan with Boc-L-Val-OH is carried out in a manner similar to that described in general procedure for the synthesis of dipeptide prodrugs of dextrorphan (Scheme 5). The Boc-protected intermediate is deprotected with 4N HCl in dioxane to 3-L-Val-dextrorphan 2 HCl and then coupled with Boc-Val-OSu to give 3-(Boc-L-Val-L-Val)-dextrorphan that is subsequently deprotected with 4N HCl in dioxane to give the corresponding hydrochloride salt 7a by treatment.

3-(D-Val-D-Val)-dextrorphan 2 HCl (7b):

The synthesis of 3-(D-Val-D-Val)-dextrorphan 2 HCl is carried out in a manner similar to that described for the synthesis of 7a.

3-(L-Ala-L-Ala)-dextrorphan 2 HCl (8a):

The synthesis of 3-(L-Ala-L-Ala)-dextrorphan 2 HCl is carried out in a manner similar to that described for the synthesis of 7a.

3-(D-Ala-D-Ala)-dextrorphan 2 HCl (8b):

The synthesis of 3-(D-Ala-D-Ala)-dextrorphan 2 HCl is carried out in a manner similar to that described for the synthesis of 7a.

3-(D-Ala-D-Val)-dextrorphan 2 HCl (9a):

The synthesis of 3-(D-Ala-D-Val)-dextrorphan 2 HCl is carried out in a manner similar to that described for the synthesis of 7a.

TABLE 2
Observed Chiral Inversion of N-Acetyl-L-Amino Acids
during the Synthesis of Dextrorphan Prodrugs

| | Reaction | Unreacted | Product | Chiral |
|---|---|---|---|---|
| Amino Acid Starting Material | Time (h) | Dextrorphan | Yield | Inversion^{a} |
| N-Ac-L-valine (Ac-L-Val-OH) | 120 | 32% | 68% | Yes (50:50) |
| N-Ac-L-alanine (Ac-L-Ala-OH) | 10 | 12% | 88% | No |
| N-Ac-L-phenylalanine (Ac-L-Phe-OH) | 12 | 27% | 73% | No |
| N-Ac-L-isoleucine (Ac-L-Ile-OH) | 120 | 33% | 67% | Yes (25:75) |
| N^{α},N^{ε}-Ac-L-lysine (Ac-Lys(Ac)-OH) | 24 | 19% | 81% | No |
| N-Ac-L-glutamic acid ^{t}butyl ester | 23 | 11% | 89% | No |
| (Ac-Glu(^{t}Bu)-OH) | | | | |

^{a}At the amino acid

TABLE 3
Observed Chiral Inversion of Differently Protected L-
Valine during the Synthesis of Dextrorphan Prodrugs

| | Reaction | Unreacted | Product | Chiral |
|---|---|---|---|---|
| Amino Acid Starting Material | Time (h) | Dextrorphan | Yield | Inversion^{a} |
| N-Ac-L-valine (Ac-L-Val-OH) | 120 | 32% | 68% | Yes (50:50) |
| N-Bz-L-valine (Bz-L-Val-OH) | 72 | 30% | 70% | Yes (50:50) |
| N-Boc-L-valine (Boc-L-Val-OH) | 17 | 2% | 98% | No |
| N-phthaloyl-valine (phthaloyl-Val-OH) | 6 | 7% | 97% | Yes (50:50) |

^{a}At the amino acid
Ac = acetyl; Bz = benzoyl; Boc = tert.-butoxycarbonyl.

TABLE 4
Observed Chiral Inversion of N-Acetyl-L-Valine during the Synthesis
of Dextrorphan Prodrugs Using Different Reaction Conditions

| | Reaction | Unreacted | Product | Chiral |
|---|---|---|---|---|
| Reaction Condition | Time (h) | Dextrorphan | Yield | Inversion^{a} |
| 1.1 eq. BOP, 1.1 eq. HOBT, | 120 | 32% | 68% | Yes |
| 2.5 eq. DIPEA, DMF [0.1 mmol] | | | | |
| 1.5 eq. DCC, 0.1 eq. DMAP, | 96 | 24% | 76% | Yes |
| DCM [0.1 mmol] | | | | |
| 1.1 eq. TBTU, 2.5 eq. DIPEA, | 70 | 42% | 58% | Yes |
| DMF [0.1 mmol] | | | | |

^{a}At the amino acid
eq. = equivalents; BOP = benzotriazole-1-yl-oxy-tris-(dimethylamino)-phosphonium hexafluorophosphate; HOBT = 1-hydroxybenzotriazole; DIPEA = N,N-diisopropylethylamine; DMF = dimethylformamide; DMAP = 4-(dimethylamino)pyridine; DCM = dichloromethane; TBTU = 2-(1H-benzotriazole-1-yl)-1,1,3,3-tetramethylaminium tetrafluoroborate.

### Example 2-Oral Administration of Chiral Amino Acid Ester Prodrugs of Dextrorphan

Solutions of dextrorphan tartrate (comparator) and amino acid prodrugs of dextrorphan in water were administer in Sprague-Dawley rats via oral gavage at doses equimolar to 6 mg/kg dextrorphan tartrate. Blood samples were collected at 0.25, 0.5, 1, 2, 4, and 6 hours postdose. Plasma concentrations of dextrorphan were determined using tandem liquid chromatography-mass spectrometry (LC-MS/MS).

The Area Under the Curve (AUC), maximum blood concentration (Cₘₐₓ) and time to maximum blood concentration Tₘₐₓ for several aspects of the present technology are compared to dextrorphan in Table 5 below:

TABLE 5
Mean Pharmacokinetic Parameters after Oral Administration in Rats.

| | | | | Dextrorphan Control^{b} | | | | |
|---|---|---|---|---|---|---|---|---|
| | AUC_{0-6 h} | Cₘₐₓ | Tₘₐₓ | AUC_{0-6 h} | Cₘₐₓ | Tₘₐₓ | Exposure Ratio^{c} | |
| Compound^{a} | [ng/mL × h] | [ng/mL] | [h] | [ng/mL × h] | [ng/mL] | [h] | AUC | Cₘₐₓ |
| 3-(N-Ac-L- | 27.7 | 8.0 | 2.8 | 63.1 | 44.6 | 2.6 | 44% | 18% |
| Phe)- | | | | | | | | |
| dextrorphan | | | | | | | | |
| 3-(N-Ac-D- | 19.1 | 4.7 | 5.0 | 63.1 | 44.6 | 2.6 | 30% | 11% |
| Phe)- | | | | | | | | |
| dextrorphan | | | | | | | | |
| 3-(N-Ac-L- | 27.4 | 13.9 | 2.2 | 33.5 | 12.2 | 1.4 | 82% | 114% |
| Ile)- | | | | | | | | |
| dextrorphan | | | | | | | | |
| 3-(N-Ac-D- | 294.6 | 81.9 | 2.9 | 33.5 | 12.2 | 1.4 | 880% | 672% |
| Ile)- | | | | | | | | |
| dextrorphan | | | | | | | | |
| 3-(N-Ac-L- | 56.7 | 29.8 | 1.1 | 93.0 | 68.0 | 0.7 | 61% | 44% |
| Lys(Ac))- | | | | | | | | |
| dextrorphan | | | | | | | | |
| 3-(N-Ac-D- | 18.0 | 4.7 | 3.9 | 93.0 | 68.0 | 0.7 | 19% | 7% |
| Lys(Ac))- | | | | | | | | |
| dextrorphan | | | | | | | | |
| 3-(N-Ac-L- | 31.2 | 12.6 | 2.8 | 84.0 | 70.9 | 0.6 | 37% | 18% |
| Val)- | | | | | | | | |
| dextrorphan | | | | | | | | |
| 3-(N-Ac-D- | 467.4 | 127.7 | 3.0 | 84.0 | 70.9 | 0.6 | 556% | 180% |
| Val)- | | | | | | | | |
| dextrorphan | | | | | | | | |
| 3-(N-Ac-L- | 29.6 | 18.2 | 0.7 | 73.5 | 52.1 | 0.7 | 40% | 35% |
| Ala)- | | | | | | | | |
| dextrorphan | | | | | | | | |
| 3-(N-Ac-D- | 30.8 | 16.5 | 1.6 | 73.5 | 52.1 | 0.7 | 42% | 32% |
| Ala)- | | | | | | | | |
| dextrorphan | | | | | | | | |
| 3-(N-Ac-L- | 18.4 | 4.8 | 1.8 | 43.0 | 35.0 | 0.3 | 43% | 14% |
| Glu)- | | | | | | | | |
| dextrorphan | | | | | | | | |
| 3-(N-Ac-D- | 12.7 | 3.5 | 2.4 | 43.0 | 35.0 | 0.3 | 30% | 10% |
| Glu)- | | | | | | | | |
| dextrorphan | | | | | | | | |
| 3-(L-Val-L- | 78.5 | 98.9 | 0.5 | 92.1 | 77.4 | 0.5 | 85% | 128% |
| Val)- | | | | | | | | |
| dextrorphan | | | | | | | | |
| 3-(L-Ala-L- | 88.3 | 87.3 | 0.8 | 59.9 | 40.1 | 0.5 | 147% | 217% |
| Ala)- | | | | | | | | |
| dextrorphan | | | | | | | | |
| 3-(D-Ala-D- | 24.7 | 9.6 | 2.1 | 42.0 | 22.5 | 3.5 | 59% | 43% |
| Ala)- | | | | | | | | |
| dextrorphan•2 | | | | | | | | |
| HCl | | | | | | | | |

^{a}Compounds were dosed as hydrochloride salt.
^{b}Dextrorphan was dosed as tartrate salt.
^{c}Exposure Ratio is the ratio of the respective PK parameter of the compound and the PK parameter of dextrorphan control expressed as percent (%).

The results showed that the oral bioavailability of dextrorphan differed dramatically between the L- and D-amino acid diastereomers of certain prodrugs but not others. Moreover, the magnitude and direction of the difference in dextrorphan exposure varied depending on the amino acid(s) conjugated with dextrorphan.

For example, total dextrorphan exposure as measured by AUC₀₋₆ₕ and peak dextrorphan exposure as measured by Cₘₐₓ were lower following dosing of the prodrugs with phenylalanine (Phe), alanine (Ala), or glutamic acid (Glu) attached to dextrorphan when compared to dextrorphan tartrate, and the exposure levels were similar regardless whether the L- or D-isomer of the respective amino acid was bound to dextrorphan.

Comparing the L- and D-conjugates of N—Ac-Ala and N—Ac-valine (Val) to dextrorphan tartrate exemplifies the unpredictability of amino acid conjugation. The Ala amino acid side chain is a methyl (—CH₃) group, while the Val amino acid side chain is an isopropyl group (—CH(CH₃)₂). The β-carbon in N—Ac-Val is disubstituted but monosubstituted in N—Ac-Ala. Both the L- and D-conjugates of N—Ac-Ala have AUCs of 40% and 32% of dextrorphan tartrate, respectively, and have Cₘₐₓ of 35% and 32% of dextrorphan, respectively. 3-(N—Ac-L)-Val-dextrorphan also has a similar bioavailability (AUC=37%, Cₘₐₓ=18%) as the N—Ac-L-Ala conjugate; changing the amino acid side chain from a methyl group to an isopropyl group seemingly has a minor impact on bioavailability for the L-diastereomer of N—Ac-Ala and N—Ac-Val. However, the D-N—Ac-Val conjugate has an unexpectedly large bioavailability (AUC=556%, Cₘₐₓ=180%) compared to the N—Ac-D-Ala conjugate (42%, 32% respectively) and the N—Ac-L-Val conjugate.

The amino acid side chains in isoleucine (Ile) and valine (Val) differ by an additional carbon on the Ile chain (isobutyl structure). However, the N—Ac-L-Ile conjugate has higher bioavailability (82% AUC, 114% Cₘₐₓ) whereas the N—Ac-L-Val conjugate has a 37% AUC and 18% Cₘₐₓ compared to dextrorphan tartrate. However, similar to the N—Ac-D-Val conjugate, the N—Ac-L-Ile conjugate shows a significant increase in bioavailability compared to dextrorphan tartrate (880% AUC, 682% Cₘₐₓ).

More unexpectedly, the L-Ala-L-Ala conjugate showed a large increase in bioavailability compared to the Ac-L-Ala conjugate. The Ac-L-Ala conjugate showed a decreasing bioavailability compared to dextrorphan tartrate. Furthermore, the D-Ala-D-Ala conjugate also showed decreased bioavailability (59% AUC, 43% Cₘₐₓ) compared to dextrorphan tartrate. Yet the L-Ala-L-Ala conjugate produced greater bioavailability (147% AUC, 217% Cₘₐₓ).

Still more unexpectedly, the dextrorphan AUC₀₋₆ₕ was more than 5 and 8 times higher after dosing of 3-(N—Ac-D-Val)-dextrorphan·HCl and 3-(N—Ac-D-Ile)-dextrorphan·HCl, respectively, when compared to dextrorphan tartrate. However, the AUC₀₋₆ₕ of 3-(N—Ac-L-Val)-dextrorphan·HCl was significantly lower compared to dextrorphan tartrate, while the AUC₀₋₆ₕ of 3-(N—Ac-L-Ile)-dextrorphan·HCl was only slightly lower compared to dextrorphan tartrate. The dextrorphan Cₘₐₓ was about 1.8 times higher after administration of 3-(N—Ac-D-Val)-dextrorphan·HCl, but over 6 times higher after administration of 3-(N—Ac-D-Ile)-dextrorphan·HCl when compared to dextrorphan tartrate. Conversely, the Cₘₐₓ for 3-(N—Ac-L-Val)-dextrorphan·HCl was less than 20% of the Cₘₐₓ for dextrorphan tartrate, while the Cₘₐₓ for 3-(N—Ac-L-Ile)-dextrorphan·HCl was slightly higher compared to dextrorphan tartrate.

Also unexpectedly, the oral bioavailability of 3-(N—Ac-D-Lys(Ac))-dextrorphan·HCl was significantly lower compared to 3-(N—Ac-L-Lys(Ac))-dextrorphan·HCl in opposition to the prodrugs containing the L- or D-isomer of valine (Val) and isoleucine (Ile).

In addition, unlike the single amino acid prodrug 3-(N—Ac-L-Val)-dextrorphan·HCl, the dipeptide prodrug 3-(L-Val-L-Val)-dextrorphan·2 HCl demonstrated good oral bioavailability with slightly lower dextrorphan AUC₀₋₆ₕ and higher dextrorphan Cₘₐₓ compared to dextrorphan tartrate. However, the 3-(D-Val-D-Val)-dextrorphan·2 HCl isomer had significantly increased bioavailability compared to 3-(L-Val-L-Val)-dextrorphan·2 HCl with about 4.5- and 2.7 fold increase in AUC₀₋₆ₕ and Cₘₐₓ, respectively, when compared to dextrorphan tartrate.

The dextrorphan exposure levels after oral administration of 3-(D-Ala-D-Ala)-dextrorphan·2 HCl were only slightly higher compared to 3-(Ac-D-Ala)-dextrorphan·HCl and thus, lower relative to dextrorphan tartrate. Contrarily, dextrorphan AUC₀₋₆ₕ and Cₘₐₓ of 3-(L-Ala-L-Ala)-dextrorphan·2 HCl were much higher compared to 3-(Ac-L-Ala)-dextrorphan·HCl and about 1.5 and over 2 times higher, respectively, compared to dextrorphan tartrate.

The mixed peptide 3-(D-Ala-D-Val)-dextrorphan·2 HCl showed superior dextrorphan bioavailability compared to dextrorphan tartrate, 3-(L-Val-L-Val)-dextrorphan·2 HCl, and 3-(D-Ala-D-Ala)-dextrorphan·2 HCl, but lower dextrorphan exposure compared to 3-(D-Val-D-Val)-dextrorphan·2 HCl and 3-(L-Ala-L-Ala)-dextrorphan·2 HCl.
1. A compound having a structure of Formula I:

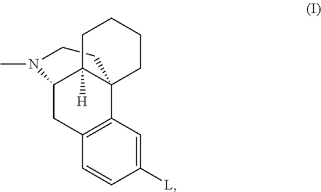

where L is A¹-G¹ or A¹-A²-G², and A¹ and A² are independently selected optically active amino acids, wherein at least one of A¹ and A² has a chiral center, G¹ is an acyl group, and G² is an acyl group or absent; or a pharmaceutically acceptable salt thereof.
2. The compound according to 1, where the at least one chiral amino acid is a standard amino acid, a non-standard amino acid, or a synthetic amino acid.
3. The compound according to 2, wherein the at least one chiral amino acid is a standard amino acid.
4. The compound according to 3, wherein the standard amino acid is selected from the group consisting of isoleucine, valine, or alanine.
5. The compound according to 4, wherein the standard amino acid is selected from D-isoleucine, D-valine, and L-alanine.
6. The compound according to 1, wherein A¹ is L-alanine and A² is L-alanine.
7. The compound according to 1, wherein A¹ is D-valine and A² is D-valine.
8. The compound according to 1, wherein the compound of Formula I has a structure of Formula II:

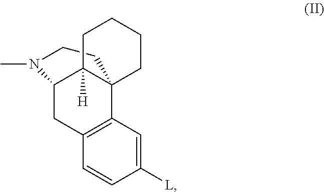

where L is A¹-G¹, A¹ is an optically active amino acids, wherein the optically active amino acid has a chiral center, and G¹ is an acyl group; or a pharmaceutically acceptable salt thereof.
9. The compound according to 8, where the chiral amino acid is a standard amino acid, a non-standard amino acid, or a synthetic amino acid.
10. The compound according to 9, wherein the chiral amino acid is a standard amino acid.
11. The compound according to 10, wherein the standard amino acid is selected from the group consisting of isoleucine, valine, or alanine.
12. The compound according to 11, wherein the standard amino acid is selected from D-isoleucine, D-valine, and L-alanine.
13. The compound according to 1, wherein the compound of Formula I has a structure of Formula III:

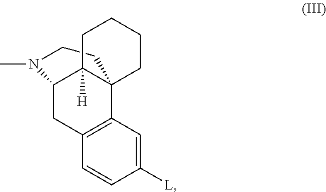

where L is A¹-A²-G², and A¹ and A² are independently selected optically active amino acids, wherein at least one of A¹ and A² has a chiral center, and G² is an acyl group or absent; or a pharmaceutically acceptable salt thereof.
14. The compound according to 13, where the at least one chiral amino acid is a standard amino acid, a non-standard amino acid, or a synthetic amino acid.
15. The compound according to 14, wherein the at least one chiral amino acid is a standard amino acid.
16. The compound according to 13, wherein A¹ is L-alanine and A² is L-alanine.
17. The compound according to 13, wherein A¹ is D-valine and A² is D-valine.
18. A composition comprising: a therapeutically effective amount of a compound having a structure of Formula I:

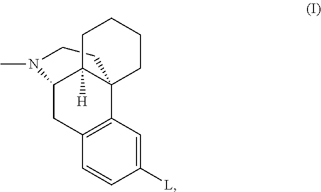

where L is A¹-G¹ or A¹-A²-G², and A¹ and A² are independently selected optically active amino acids, wherein at least one of the A¹ and/or A² has a chiral center, G¹ is an acyl group, and G² is an acyl group or absent; or a pharmaceutically acceptable salt thereof.
19. The composition according to 18, where the chiral amino acid is a standard amino acid, a non-standard amino acid, or a synthetic amino acid.
20. The composition according to 19, wherein the chiral amino acid is a standard amino acid.
21. The composition according to 20, wherein the standard amino acid is selected from the group consisting of isoleucine, valine, or alanine.
22. The composition according to 21, wherein the standard amino acid is selected from D-isoleucine, D-valine, and L-alanine.
23. The composition of 19-22, wherein the therapeutically effective amount of the compound of Formula I ranges from about 0.1 mg to about 2000 mg per day.
24. The composition of 19-23, wherein the composition further comprises at least one excipient . . . .
25. The composition of 19-24, wherein the composition is an oral dosage formulation.
26. The composition of 25, wherein the oral dosage formulation is a liquid dosage formulation selected from the group consisting of syrups, emulsions or suspensions.
27. The composition of 25, wherein the oral dosage formulation is a solid dosage formulation selected from the group consisting of a sublingual, a gummy, a chewable tablet, a rapidly dissolving tablet, a tablet, a capsule, a caplet, a troche, a lozenge, an oral powder, a thin strip, an oral thin film (OTF), and an oral strip
28. The composition of 18, wherein the compound of Formula I has a structure of Formula II:

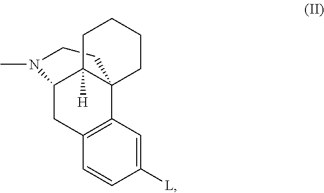

where L is A¹-G¹, and A¹ is an optically active amino acids, wherein the optically active amino acid has a chiral center, G¹ is an acyl group, or a pharmaceutically acceptable salt thereof.
29. The composition according to claim 28, where the chiral amino acid is a standard amino acid, a non-standard amino acid, or a synthetic amino acid.
30. The composition according to 29, wherein the chiral amino acid is a standard amino acid.
31. The composition according to 30, wherein the standard amino acid is selected from the group consisting of isoleucine, valine, or alanine.
32. The composition according to 31, wherein the standard amino acid is selected from D-isoleucine, D-valine, and L-alanine.
33. The composition of 28-32, wherein the therapeutically effective amount of the compound of Formula I ranges from about 0.1 mg to about 2000 mg per day.
34. The composition of 28-33, wherein the composition further comprises at least one excipient selected from the group consisting of antiadherents, binders, coatings, disintegrants, gel forming agents, fillers, flavors and colors, glidants, lubricants, preservatives, sorbents and sweeteners.
35. The composition of 34, wherein the composition is an oral dosage formulation.
36. The composition of 35, wherein the oral dosage formulation is a liquid dosage formulation selected from the group consisting of syrups, emulsions or suspensions.
37. The composition of 35, wherein the oral dosage formulation is a solid dosage formulation selected from the group consisting of a sublingual, a gummy, a chewable tablet, a rapidly dissolving tablet, a tablet, a capsule, a caplet, a troche, a lozenge, an oral powder, a thin strip, an oral thin film (OTF), and an oral strip.
38. The composition of 18, wherein the compound of Formula I has a structure of Formula III:

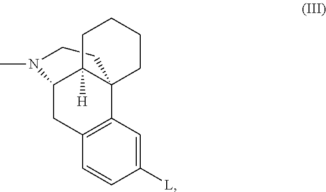

where L is A¹-A²-G², and A¹ and A² are independently selected optically active amino acids, wherein at least one of A¹ and/or A² has a chiral center, and G² is an acyl group or absent; or a pharmaceutically acceptable salt thereof.
39. The composition according to 38, where the at least one chiral amino acid is a standard amino acid, a non-standard amino acid, or a synthetic amino acid.
40. The composition according to 39, wherein the at least one chiral amino acid is a standard amino acid.
41. The composition according to 38, wherein A¹ is L-alanine and A² is L-alanine.
42. The composition according to 38, wherein A¹ is D-valine and A² is D-valine.
43. The composition of 38-42, wherein the therapeutically effective amount of the compound of Formula I ranges from about 0.1 mg to about 2000 mg per day.
44. The composition of 38-42, wherein the composition further comprises at least one excipient selected from the group consisting of antiadherents, binders, coatings, disintegrants, gel forming agents, fillers, flavors and colors, glidants, lubricants, preservatives, sorbents and sweeteners.
45. The composition of 44, wherein the composition is an oral dosage formulation.
46. The composition of claim 45, wherein the oral dosage formulation is a liquid dosage formulation selected from the group consisting of syrups, emulsions or suspensions.
47. The composition of 45, wherein the oral dosage formulation is a solid dosage formulation selected from the group consisting of a sublingual, a gummy, a chewable tablet, a rapidly dissolving tablet, a tablet, a capsule, a caplet, a troche, a lozenge, an oral powder, a thin strip, an oral thin film (OTF), and an oral strip.
48. A method of manufacturing a chiral amino acid ester prodrug of dextrorphan having a structure of Formula II:

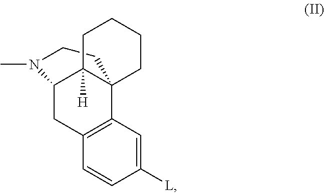

where L is A¹-G¹, A¹ is an optically active amino acids, wherein the optically active amino acid has a chiral center, and G¹ is an acyl group, and the method comprising: reacting a protected diestereomerically pure amino acid with dextrorphan in a solvent in the presence of a base for form a protected amino acid ester intermediate; and reacting the protected amino acid ester intermediate with an acid to produce a compound of Formula II.
49. The method of 48, wherein the protected disetereomerically pure amino acid is protected by a protecting group selected from the group consisting of acetyl (Ac), tert-butyoxycarbonyl (Boc), tert-butyl (′Bu), benzyloxycarbonyl (Cbz), p-methoxybenzylcarbonyl (Moz), 9-fluorenylmethyloxycarbonyl (Fmoc), benzyl (Bn), benzoyl (Bz), phthaloyl, p-methoxybenzyl (PMB), 3,4 dimethoxybenzyl (DMPM), p-methozyphenyl (PMP), tosyl (Ts), acetamide, and pthalamide.
50. The method of 49, wherein the protecting group is tert-butyoxycarbonyl (Boc).
51. The method of 48, wherein the base is selected from the group consisting of 4-methylmorpholine (NMM), 4-(dimethylamino)pyridine (DMAP), N,N-diisopropylethylamine (DIPEA), lithium bis(trimethylsilyl)amide, lithium diisopropylamide (LDA), potassium tert.-butoxide, sodium tert-butoxide, lithium tert-butoxide, sodium hydride, potassium hydride, lithium hydride, an alkoxide, and a tertiary amine.
52. The method of 48 wherein the solvent is selected from the group consisting of acetone, acetonitrile, butanol, chloroform, dichloromethane (DCM), dimethylformamide (DMF), dimethylsulfoxide (DMSO), dioxane, ethanol, ethyl acetate, diethyl ether, heptane, hexane, methanol, methyl tert.-butyl ether (MTBE), isopropanol, isopropyl acetate, diisopropyl ether, tetrahydrofuran, toluene, xylene, and water.
53. A method of manufacturing a chiral amino acid ester prodrug of dextrorphan comprising a compound having Formula III:

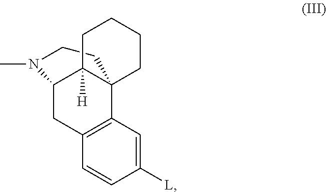

where L is A¹-A²-G², A¹ and A² are independently selected optically active amino acids, wherein at least one of A¹ and A² has a chiral center, and G² is an acyl group or absent; the method comprising: reacting a first protected diestereomerically pure amino acid with dextrorphan in a solvent in the presence of a base to produce a first intermediate; reacting the first intermediate with an acid to form a deprotected amino acid ester intermediate; reacting the deprotected amino acid ester intermediate with a second protected diestereomerically pure amino acid to produce a protected dipeptide intermediate in a second solvent in the presence of a second base; and reacting the protected dipeptide intermediate with an acid to produce a compound of Formula III.
54. The method of claim 53, wherein the protected disetereomerically pure amino acid is protected by a first protecting group selected from the group consisting of acetyl (Ac), tert-butyoxycarbonyl (Boc), tert-butyl (′Bu), benzyloxycarbonyl (Cbz), p-methoxybenzylcarbonyl (Moz), 9-fluorenylmethyloxycarbonyl (Fmoc), benzyl (Bn), benzoyl (Bz), phthaloyl, p-methoxybenzyl (PMB), 3,4 dimethoxybenzyl (DMPM), p-methozyphenyl (PMP), tosyl (Ts), acetamide, and pthalamide.
55. The method of 54, wherein the protecting group is tert-butyoxycarbonyl (Boc).
56. The method of 53, wherein the base is selected from the group consisting of 4-methylmorpholine (NMM), 4-(dimethylamino)pyridine (DMAP), N,N-diisopropylethylamine (DIPEA), lithium bis(trimethylsilyl)amide, lithium diisopropylamide (LDA), potassium tert.-butoxide, sodium tert-butoxide, lithium tert-butoxide, sodium hydride, potassium hydride, lithium hydride, an alkoxide, and a tertiary amine.
57. The method of 53 wherein the solvent is selected from the group consisting of acetone, acetonitrile, butanol, chloroform, dichloromethane (DCM), dimethylformamide (DMF), dimethylsulfoxide (DMSO), dioxane, ethanol, ethyl acetate, diethyl ether, heptane, hexane, methanol, methyl tert.-butyl ether (MTBE), isopropanol, isopropyl acetate, diisopropyl ether, tetrahydrofuran, toluene, xylene, and water.
58. A compound having a structure of Formula III:

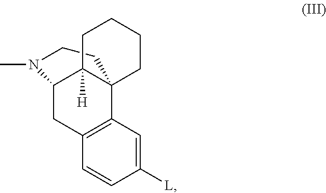

or a pharmaceutically acceptable salt thereof, where L is A¹-A²-G², and A¹ and A² are independently selected amino acids, wherein at least one of A¹ and A² has a chiral center and is optically active, and G² is an acyl group or absent; or a pharmaceutically acceptable salt thereof.
59. A compound having a structure selected from the group consisting of

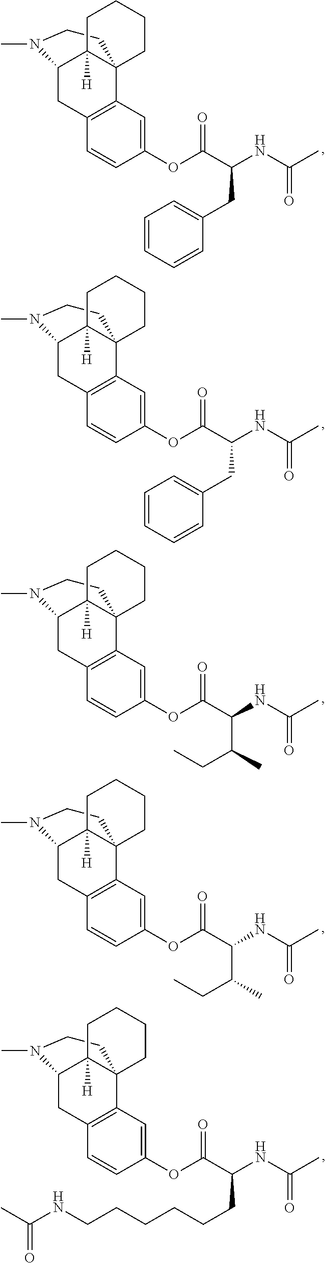

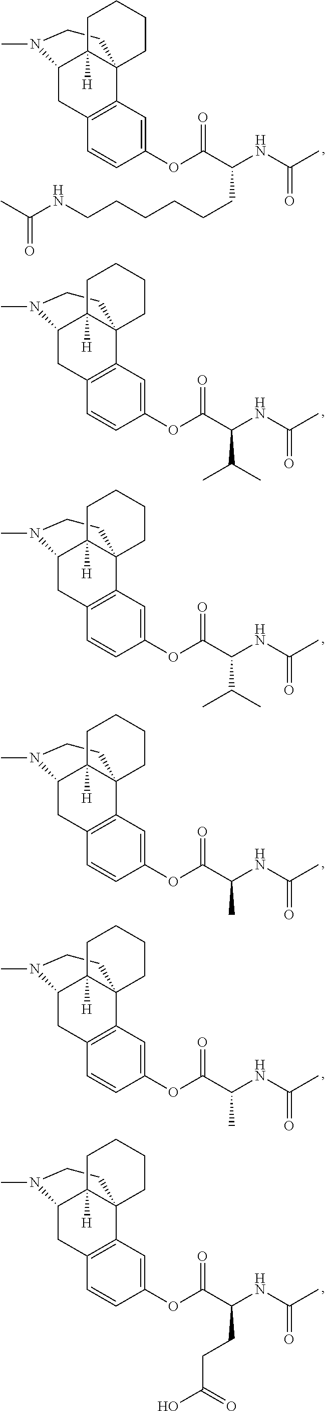

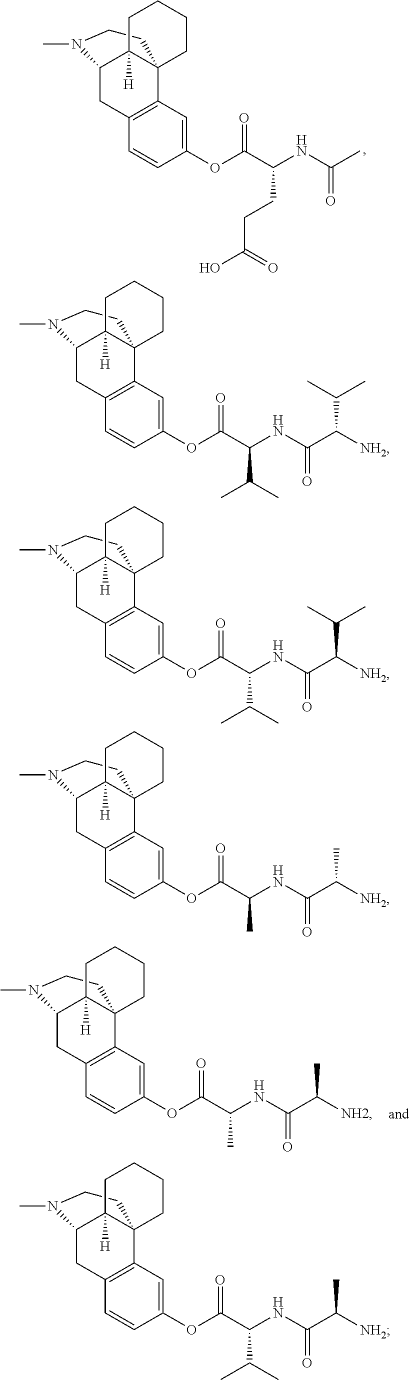

or a pharmaceutically acceptable salt thereof.

The presently described technology and its advantages will be better understood by reference to the following examples. These examples are provided to describe specific embodiments of the present technology. By providing these specific examples, it is not intended limit the scope and spirit of the present technology. It will be understood by those skilled in the art that the full scope of the presently described technology encompasses the subject matter defined by the claims appending this specification, and any alterations, modifications, or equivalents of those

1. A compound having a structure of Formula I: 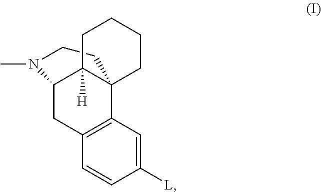
where L is A¹-G¹ or A¹-A²-G², and
A¹ and A² are independently selected optically active amino acids, wherein at least one of A¹ and A² has a chiral center,
G¹ is an acyl group, and
G² is an acyl group or absent; or
a pharmaceutically acceptable salt thereof.

2. The compound according to claim 1, where the at least one chiral amino acid is a standard amino acid, a non-standard amino acid, or a synthetic amino acid.

3. The compound according to claim 2, wherein the at least one chiral amino acid is a standard amino acid.

4. The compound according to claim 3, wherein the standard amino acid is selected from the group consisting of isoleucine, valine, or alanine.

5. The compound according to claim 4, wherein the standard amino acid is selected from D-isoleucine, D-valine, and L-alanine.

6. The compound according to claim 1, wherein A¹ is L-alanine and A² is L-alanine.

7. The compound according to claim 1, wherein A¹ is D-valine and A² is D-valine.

8. The compound according to claim 1, wherein the compound of Formula I has a structure of Formula II: 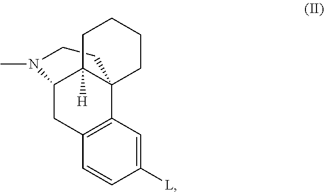
where L is A¹-G¹,
A¹ is an optically active amino acids, wherein the optically active amino acid has a chiral center, and
G¹ is an acyl group; or
a pharmaceutically acceptable salt thereof.

9. The compound according to claim 8, where the chiral amino acid is a standard amino acid, a non-standard amino acid, or a synthetic amino acid.

10. The compound according to claim 9, wherein the chiral amino acid is a standard amino acid.

11. The compound according to claim 10, wherein the standard amino acid is selected from the group consisting of isoleucine, valine, or alanine.

12. The compound according to claim 11, wherein the standard amino acid is selected from D-isoleucine, D-valine, and L-alanine.

13. The compound according to claim 1, wherein the compound of Formula I has a structure of Formula III: 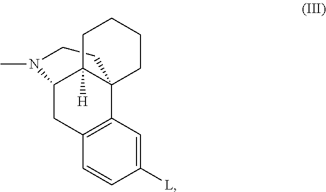
where L is A¹-A²_G², and
A¹ and A² are independently selected optically active amino acids, wherein at least one of A¹ and A² has a chiral center, and
G² is an acyl group or absent; or
a pharmaceutically acceptable salt thereof.

14. The compound according to claim 13, where the at least one chiral amino acid is a standard amino acid, a non-standard amino acid, or a synthetic amino acid.

15. The compound according to claim 14, wherein the at least one chiral amino acid is a standard amino acid.

16. The compound according to claim 13, wherein A¹ is L-alanine and A² is L-alanine.

17. The compound according to claim 13, wherein A¹ is D-valine and A² is D-valine.

18-47. (canceled)

48. A method of manufacturing a chiral amino acid ester prodrug of dextrorphan having a structure of Formula II: 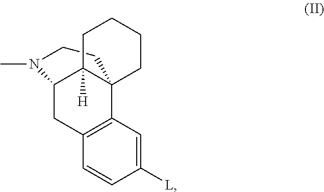
where L is A¹_G¹,
A¹ is an optically active amino acids, wherein the optically active amino acid has a chiral center, and
G¹ is an acyl group, and
the method comprising:
reacting a protected diestereomerically pure amino acid with dextrorphan in a solvent in the presence of a base for form a protected amino acid ester intermediate; and
reacting the protected amino acid ester intermediate with an acid to produce a compound of Formula II.

49-52. (canceled)

53. A method of manufacturing a chiral amino acid ester prodrug of dextrorphan comprising a compound having Formula III: 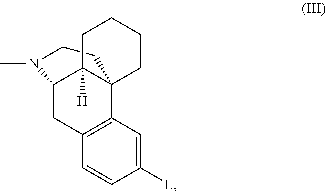
where L is A¹-A²-G²,
A¹ and A² are independently selected optically active amino acids, wherein at least one of A¹ and A² has a chiral center, and
G² is an acyl group or absent;
the method comprising:
reacting a first protected diestereomerically pure amino acid with dextrorphan in a solvent in the presence of a base to produce a first intermediate;
reacting the first intermediate with an acid to form a deprotected amino acid ester intermediate;
reacting the deprotected amino acid ester intermediate with a second protected diestereomerically pure amino acid to produce a protected dipeptide intermediate in a second solvent in the presence of a second base; and
reacting the protected dipeptide intermediate with an acid to produce a compound of Formula III.

54-59. (canceled)
